(19) United States
(12) Patent Application Publication
(10) Pub. No.: US 2004/0146866 A1
(43) Pub. Date: Jul. 29, 2004

(54) Quantitative multiplex detection of nucleic acids

(76) Inventor: Guoliang Fu, Oxford (GB)

Correspondence Address: Guoliang Fu, 24 Hayes Close, Marston, Oxford, OX3 0DZ, GB

(21) Appl. No.: 10/349,780

(22) Filed: Jan. 24, 2003

(51) Int. Cl. C12Q 1/68; C07H 21/04; C12P 19/34

(52) U.S. Cl. 435/6; 435/91.2; 536/24.3

(57) ABSTRACT

Methods are provided for quantitative multiplex detection of nucleic acids. Methods of the invention are useful for genotyping mutations, especially single nucleotide polymorphisms (SNPs), for analyzing gene expression profiles, genomic methylation patterns and any specific nucleic acids from any source.

### CROSS-REFERENCE TO RELATED APPLICATIONS

This application claims priority to and benefit of U.S. application No. 60/350,372, filed Jan. 24, 2002, the full disclosure of which is incorporated herein by reference.

### FEDERALLY SPONSORED RESEARCH

Not applicable

### SEQUENCE LISTING

Submitted

### TECHNICAL FIELD OF THE INVENTION

This invention relates to multiplex amplification and detection methods useful for genotyping mutations, especially single nucleotide polymorphisms, and for analyzing differential gene expression profiles, genomic methylation patterns and any specific nuclei acids from any source.

### BACKGROUND OF THE INVENTION

The draft human genome has been sequenced. It is estimated that the human genome contains over 30,000 genes, about 15-30% of which are active in any given tissue. Such large numbers of expressed genes make it difficult to track changes in expression patterns by available techniques. The sequenced human genome also revealed a rich source of Single Nucleotide Polymorphisms (SNPs), which are the most common type of genetic variation present in the human genome and are the foundation of powerful complex trait and pharmacogenomics analyses.

Most of the currently used methods for gene expression profiling and SNP genotyping require amplification of the target DNA by the polymerase chain reaction (PCR) technique. SNP and gene expression analysis involves the analysis of large complex fragments and this is achieved by multiplex PCR (the simultaneous amplification of different target DNA sequences in a single PCR reaction). Results obtained with multiplex PCR however are often complicated by artifacts of the amplification procedure. These include false negative results due to reaction failure and false-positive results (such as amplification of spurious products) due to non-specific priming events. Since the possibility of non-specific priming increases with each additional primer pair, conditions must be modified as necessary as individual primer sets are added.

Many multiplex PCR procedures have been presented. Primers for as-short-as-possible amplicons with similar PCR-annealing temperatures were designed; no more than eight fragments could be amplified per PCR mixture reproducibly (Pastinen et.al. 2000). The number of fragments that previously have been successfully and reproducibly amplified by multiplex PCR followed by accurate genotyping on microarrays, range from four to ten fragments per PCR reaction (Hacia et al. 1998a; Pastinen et al. 1998a), whereas multiplex PCRs of more than 40 fragments can be performed at the cost of significantly reduced accuracy and success in the genotype assignment (Wang et al. 1998; Hacia et al. 1999b; Cho et al. 1999).

Other attempts have been made to overcome some of the disadvantages associated with multiplex PCR. One method is based on an additional pre-amplification step yielding a long DNA fragment, which is then used as a template for reamplification of short amplicons (Li et al. Nucleic Acids Research 24: 538, 1996). This approach however is not very robust for amplifying multiple targets from different parts of complex genomes. Another PCR-based multiplex approach for large-scale genotype analysis has been developed by Lin et.al. (Proc. Natl. Acad. Sci. USA, 1996). In this approach, the multiplex amplification procedure consists of three PCR rounds. Shuber (U.S. Pat. No. 5,882,856) and Brownie et.al. (Nucleic acid Research, 1997, 23 3235-3241) described similar approaches that use universal sequences tagged primers for multiplex amplification. Still a lot of efforts of optimization are needed in these approaches, and the reactions may not quantitatively amplify all targets.

The advent of multiplexed SNP-based genetic analysis has underscored a need for simple and accurate genotyping methods that can accommodate thousands of loci with economy of cost and consumption of sample DNA. Current state-of-the-art in SNP analysis involves pre-amplification of genomic DNA (by PCR), followed by SNP genotyping with an allele discrimination method such as DNA cleavage, ligation, single base extension or hybridization. A common drawback for most of high throughput technologies is that a prior PCR amplification of each target is often needed, which is difficult, time consuming and expensive.

Thus, the multiplex PCR amplification remains a rate-limiting factor for developing truly high throughput systems for genotyping, gene expression profiling, methylation profiling and for detecting other nucleic acid targets.

The present invention provides quantitative multiplex amplification methods which if coupled with a detection system for example a gel or capillary electrophoresis system can be used for high throughput SNP genotyping, gene expression profiling, DNA methylation profiling and detecting multiple different nucleic acid targets.

### SUMMARY OF THE INVENTION

The present invention provides multiplex amplification methods for quantitatively detecting and genotyping specific nucleic acid sequences in biological samples. Methods disclosed herein may be used to detect and genotype mutations, analyze differential gene expression, differential genomic methylation patterns and quantitatively detect and measure the amount and presence of any specific nucleic acid of interest. Methods are useful for establishing individualized genetic profiles, and also useful for identifying nucleic acid of an invading disease-causing microorganism.

In the present invention, the multiplex amplification methods can be coupled with any detection system, for example, gel or capillary electrophoresis systems. The methods can comprise steps of initial reactions of primer extension or amplification, restriction enzyme digestion, random ligation, final amplification and detection (FIG. 1). By utilizing these combined steps, the methods of the invention allow nearly proportional amplification of different targets. The methods also are designed to eliminate certain types of amplification biases, which occur during conventional PCR amplification, arising out of different efficiency of priming and polymerization on different sequence context and length. For the detection process, means of detection known in the art can be used. Preferably, gel electrophoresis and capillary array electrophoresis are used to separate fragments that are distinguishable by sizes and /or detectable labels. In one embodiment of the invention, for detection of amplified nucleic acid products, the dNTPs, primers and single stranded from final amplification products are eliminated by any means of digestion known in the art, for example, incubating with shrimp alkaline phosphatase and exonuclease I. Subsequently, the products are digested by restriction enzyme, and analyzed on a gel directly. Alternatively, the digested products can be labeled by polymerase extension with dye labeled terminators (FIG. 1).

In one embodiment of the invention, for genotyping and detecting multiple mutations or SNPs (FIG. 2), reaction mixtures comprise target nucleic acid sample and a set or sets of multiple primers. Reverse primers can be conventional primers containing complementary portion only, or preferably comprising a 3′ complementary portion and a 5′ non-complementary portion. The allele-specific forward primer comprises two portions: a 3′ complementary portion and a 5′ non-complementary portion. The complementary portions of allele-specific forward primers comprise 3′ends which are complementary to either allele at mutation or polymorphism sites. The non-complementary portions generally contain specific sequence elements that are useful or essential for particular embodiments. In this embodiment of the invention, an allele- or gene-specific restriction site is incorporated into 5′ region of and preferably immediately adjacent to the complementary portion of each allele-specific forward primer. As in most of embodiments, wherein a common restriction site is incorporated into primers, it is preferred that in this embodiment the common restriction site is incorporated at a few bases 5′ of the allele- or gene-specific restriction sites. The common restriction site is referred to as first restriction site (its cognate enzyme is named first restriction enzyme); the allele- or gene-specific restriction site is referred to as second restriction site (its cognate restriction enzyme is named second restriction enzyme). It is designed that allele-specific primers comprise different second restriction sites which are specific for each allele. In other words, one allele-specific primer with one SNP nucleotide is tagged by one restriction site (for example Msp I), whereas another allele-specific primer with another SNP nucleotide is tagged by another restriction site (for example Dpn II). In this way, the sequences of two allele-specific primers differ in their 3′ end nucleotides and the second restriction sites that are useful for the detection of particular allele or SNPs. Alternatively, if the same second restriction enzyme site (for example, Dpn II site) is incorporated into the primers, their locations can be different between the two allele-specific primers by several bases, preferably 1 to 9 bases. In this way, the two allele-specific primers differ in the locations of second restriction sites that result in length difference in the final amplification products if digested on the second restriction sites. In this embodiment, the allele-specific primers comprise the same non-complementary portion sequences except for the second restriction sites. A link sequence between first and second restriction sites can contain any sequence. The reverse primers or allele-specific forward primers can be tagged by capture moieties, for example biotin.

In the above embodiment, a set of reverse and allele-specific forward primers targeting multiple mutation or SNPs is mixed with target nucleic acid sample under appropriate primer extension or amplification conditions. Alternatively, different sets of reverse and allele-specific forward primers are mixed with target nucleic acid samples in separate reactions under appropriate primer extension or amplification conditions. Primer extension is performed once or more than once with the same or different set of multiple primers; or amplification is performed once or more than once with the same or different set of multiple primers each for a few cycles which can be 1 to 30 cycles, or more preferably 3 to 15 cycles. Optionally, the resulted products are immobilized to a solid support via binding capture moiety. After subsequent purification, the DNA product is restriction digested with the first restriction enzyme or with the second restriction enzyme if the first restriction sites are not incorporated into the allele-specific forward primers. Digested DNA products are joined with a DNA ligase to create randomly ligated nucleic acid fragments. The ligated DNAs are amplified using reverse primers or the universal primes having sequences identical or homologous to the non-complementary portions of the reverse primers. The amplified products can be detected by any method known in the art. In one method for the detection, the dNTPs and primers from final amplification products are eliminated by digestion. After purification, the DNA products are digested with the second restriction enzymes, then are extended with a DNA polymerase in the presence of dye labeled terminators. Subsequently, the products are analyzed on a gel or capillary electrophoresis.

In another embodiment of the invention, for genotyping and detecting multiple mutations or SNPs (FIG. 3), reaction mixtures comprise target nucleic acid samples and a set or sets of multiple primers. The sequences of non-complementary portions of the two allele-specific forward primers are different, for example, one allele-specific primer having T7 promoter sequence and another allele-specific primer having T3 promoter sequence. A common restriction site is designed or chosen for all targets either on primer sequences or on target sequences. The reverse primers are tagged with capture moieties, or preferably universal primers having sequences identical or homologous to the non-complementary portions of reverse primers are tagged with capture moieties. The target nucleic acid sample is mixed with the sets of multiple primers under appropriate primer extension or amplification conditions. Primer extension is performed once or more than once with the same or different set of multiple primers; or amplification is performed once or more than once with the same or different set of multiple primers each for a few cycles which can be 1 to 30 cycles, or more preferably 3 to 15 cycles. Optionally, the resulted products are immobilized to a solid support via binding capture moiety. After subsequent purification, the DNA products are digested on the restriction sites. The digested products are then joined with a DNA ligase. The ligated products are amplified using universal primers having the sequences identical or homologous to non-complementary portions of forward primers, for example T7 and T3 primers. It is preferred that the universal primers are tagged by different fluorescence dyes. The amplified products are detected by a detection method. For example, the DNA can be digested with the restriction enzyme and analyzed on a sequencing gel.

In yet another embodiment of the invention, methods are designed for quantitative detection of multiple nucleic acid target sequences, for example analyzing gene expression profiles, DNA methylation patterns, disease-causing microorganisms and virus nucleic acids. One reverse primer and one forward primer for each target sequence are included in a set of multiple primers. A common restriction site is designed or chosen for all targets either on primer sequences or on target sequences. The reverse primers are tagged by capture moiety, or preferably universal primers having sequences identical or homologous to the non-complementary portion sequences of reverse primers are tagged by a capture moiety. The universal primers with capture moiety are incorporated into products at some stage of reaction. A set or sets of multiple primers are mixed with target nucleic acid samples in reactions under appropriate primer extension or amplification conditions. Alternatively, different sets of multiple primers are mixed with target nucleic acid samples in separate reactions under appropriate primer extension or amplification conditions. An initial reaction of primer extension is performed once (FIG. 5) or an initial amplification is performed for a few cycles which can be 1 to 30 cycles, or more preferably 3 to 15 cycles (FIG. 4). The resulted products can be mixed with another set of multiple primers under appropriated primer extension (FIG. 5) or amplification conditions (FIG. 4), in which primer extension or amplification can be performed once or a few cycles which can be 1 to 30 cycles, or more preferably 3 to 15 cycles. Optionally, the resulted products are immobilized to a solid support via binding capture moiety. After subsequent purification, the DNA products are digested on the restriction sites. The digested products or a part of digested products with or without capture moieties are then joined with a DNA ligase. If desirable, the digested products or parts of digested products from separate reactions are ligated together. The ligated products are amplified using forward primers or preferably the universal primers having sequences identical or homologous to the non-complementary portion sequences of forward primers. The amplified products are detected by any method known in the art.

In another aspect, the invention encompasses methods for high-throughput genetic screening. The method, which allows the rapid and simultaneous detection of multiple defined target DNA sequences in DNA samples obtained from a multiplicity of individuals, is carried out by simultaneously amplifying many different target sequences from a large number of patient DNA samples.

In yet another aspect, the present invention provides single-stranded oligonucleotide primers for detection of a target DNA sequence. The 5′ non-complementary portion of the primer comprises at least one restriction enzyme site, which acts as detection marker in a process of detecting the target nucleic acid sequence. A detection signal generated from enzymatic manipulation on the restriction site in a reaction product is indicative of the presence of the target nucleic acid sequence. The restriction site can be designed to be allele-specific, gene-specific or SNP-specific.

The methods and compositions of the present invention can be applied to the diagnosis of genetic and infectious diseases, gender determination, genetic linkage analysis, and forensic studies.

### DESCRIPTION OF THE DRAWINGS

FIG. 1 is a schematic diagram of an example of multiplex amplification and detection of multiple targets in a nucleic acid sample. (1) A set or sets of multiple primers are incubated with target DNA in reactions of primer extension or amplification (also referred to as first amplification). This amplification is carried out for 1 to 15 cycles. (2) The primer extension or amplification products are digested on restriction sites with cognate restriction enzymes. Optionally, before digestion the products may be immobilized to a solid support. (3) Purification. (4) The digested and purified products are then ligated to each other using a DNA ligase. (5) The ligated products are then amplified using target specific primers or universal primers. A detection method provided in this invention using electrophoresis is shown in the detection part of this diagram. (6) dNTP, primers and single stranded products are eliminated. This step is optional. This elimination step may be carried out by any digestion and/or purification method. (7) The products are digested on the restriction sites. (8) If fluorescence dye labeled primers are used in step 5, products may be directly subjected to a detection system, for example running an electrophoresis gel. (9) If non-labeled primers are used in step 5, digested products are extended by a DNA polymerase in the presence of labeled terminator nucleotides. This step requires that in step 7 restriction digestion leaves digested DNA with 5′ protruding ends. (10) Denaturing the extended double stranded products, running the DNA on gel or capillary electrophoresis. (11) Analyzing the data, preferably analyzing the data with an aid of a computer program.

FIG. 2 is a schematic diagram of an example of detection of multiple SNPs. A target nucleic acid sample is mixed with a set of reverse and allele-specific forward primers targeting multiple mutations or SNPs under appropriate primer extension conditions. For each mutation or SNP, one reverse and two allele-specific forward primers are included in the set of multiple primers. The figure shows an example of sequences of two allele-specific primers. One allele-specific forward primer with allele nucleotide C is tagged with a restriction site Msp I in the second restriction site; another allele-specific primer with allele nucleotide T is tagged with another restriction site Dpn II. The non-complementary portions of all allele-specific forward primers contain a common first restriction enzyme site EcoR I which is incorporated at three bases 5′ of the second restriction sites. The 5′ ends of allele-specific forward primers are tagged with biotin. Primer extension or amplification is performed once or preferably a few cycles which can be 1 to 30 cycles, or more preferably 3 to 15 cycles. Optionally, the primer extension products are immobilized to a solid support via binding streptavidin coated beads. After subsequent purification and restriction digestion with the first restriction enzyme or with the second restriction enzymes if the first restriction sites are not included in the allele-specific forward primers, the digested products or parts of digested products without capture moieties are then ligated to each other with a DNA ligase. The ligated products are amplified using reverse primers or universal primers. The amplified products are detected by any method known in the art.

FIG. 3 is a schematic diagram of another example of detection of multiple SNPs. The reaction mixtures comprise target nucleic acid sample and a set of multiple primers. For each mutation or SNP, one reverse and two allele-specific forward primers are included in the set of multiple primers. The non-complementary portions of the two allele-specific forward primers have different sequences, for example T7 or T3 promoter sequences. A common restriction site is designed or chosen for all targets either on primer sequences or on target sequences. The reverse primers are tagged by a capture moiety, for example biotin. A target nucleic acid sample is mixed with a set or sets of multiple primers under appropriate primer extension conditions. Primer extension is performed once or more than once with the same or different sets of multiple primers; or amplification is performed once or more than once with the same or different sets of multiple primers each for a few cycles which can be 1 to 30 cycles, or more preferably 3 to 15 cycles. Optionally, the resulted products are immobilized to a solid support via binding capture moiety. After subsequent purification, the DNA products are digested on the restriction sites. The digested DNA are then ligated to each other with a DNA ligase. The ligated DNA are amplified using universal primers having the sequences identical or homologous to non-complementary portions of primers, for example T7 and T3 primers. It is preferred that the universal primers are labeled by different fluorescence dyes. The amplified products are detected by a detection method.

FIG. 4 is a schematic diagram of an example of quantitative amplification and detection of multiple nucleic acid target sequences, for example, analyzing gene expression profiles, DNA methylation patterns, disease-causing microorganisms and virus nucleic acids. A reaction comprises target nucleic acid sample and a set or sets of multiple primers. For each target, one reverse primer and one forward primer are included in the set of multiple primers. A common restriction site is designed or chosen for all targets either on primer sequences or on target sequences. The reverse primers can be tagged by capture moiety, for example biotin. A target nucleic acid sample is mixed with a set or sets of multiple primers under appropriate primer extension or amplification conditions. Primer extension is performed once or amplification is performed for a few cycles which can be 1 to 30 cycles. The resulted products can be mixed with another set of multiple primers under appropriated primer extension or amplification conditions, in which primer extension or amplification can be performed once or a few cycles. Optionally, the resulted products are immobilized to a solid support via binding capture moiety. After subsequent purification, the DNA products are digested on the restriction sites. The digested DNA or the part of digested DNA with or without capture moieties are then ligated to each other with a DNA ligase. The ligated products are amplified using forward primers or preferably the universal primers having sequences identical or homologous to the non-complementary portion sequences of forward primers. The amplified products are detected by any method known in the art.

FIG. 5 is a schematic diagram of an example of multiplex amplification and detection of multiple RNA transcripts. A target RNA sample is mixed with a set of multiple target specific reverse primers, random primers or oligo dT primers under appropriate reverse transcription conditions, wherein a single stranded cDNA is synthesized. Double stranded cDNA is synthesized by using a set of multiple target specific forward primers or random primers under appropriate second strand cDNA synthesis condition. Optionally, the ds cDNAs are immobilized to a solid support via binding capture moiety. The synthesized regions of ds cDNAs are pre-chosen such that all fragments contain at least one restriction site (herein Dpn II site). After subsequent purification, the ds cDNA are restriction digested. The digested ds cDNA may be purified or immobilized to remove the parts with capture moiety. The digested ds cDNA or parts of ds cDNA without capture moieties are ligated to each other by a DNA ligase under standard ligation conditions. The ligated products are amplified using universal primers having sequences identical or homologous to non-complementary portions of forward primers. The amplified products are detected by any method known in the art.

FIG. 6 and FIG. 7 are schematic diagrams of an example of multiplex amplification and detection of SNPs using allele-specific primers. Various primers and their locations are shown in FIG. 6. Examples of universal primer sequences and primer sequences for one SNP are shown in the FIG. 6.

FIG. 7 shows a detailed experimental procedure which is presented in Example 1.

FIG. 8 is an experimental result of Example 1.

FIG. 9 shows an alternative detection method of amplified EcoR I products from example presented in FIG. 6, FIG. 7, FIG. 8, and Example 1. This alternative detection method is designed to detect amplified products using fluorescence labeled terminator ddNTP and DNA extension by a DNA polymerase.

FIG. 10 is a schematic diagram of an example of multiplex amplification and detection of multiple DNA targets comprising SNPs. Various primers and their locations are shown in the figure. Three experimental procedures are presented and are described in Example 2.

FIG. 11 is an experimental result of Example 2.

### DETAILED DESCRIPTION OF THE INVENTION

To facilitate understanding of the invention, a number of terms are defined below.

A “nucleic acid”, as used herein, is a covalently linked sequence of nucleotides in which the 3′ position of the pentose of one nucleotide is joined by a phosphodiester group to the 5′ position of the pentose of the next, and in which the nucleotide residues (bases) are linked in specific sequence; i.e., a linear order of nucleotides. A “polynucleotide”, as used herein, is a nucleic acid containing a sequence that is greater than about 100 nucleotides in length. An “oligonucleotide”, as used herein, is a short polynucleotide or a portion of a polynucleotide. An oligonucleotide typically contains a sequence of about two to about one hundred bases.

“Primer” as used herein refers to an oligonucleotide, whether occurring naturally or produced synthetically, which is capable of acting as a point of initiation of synthesis when placed under conditions in which synthesis of primer extension product which is complementary to a nucleic acid strand is induced i.e., in the presence of nucleotides and an agent for polymerization such as DNA polymerase and at a suitable temperature and buffer. The primers herein are selected to be substantially complementary to the different strands of each specific sequence to be amplified. This means that the primers must be sufficiently complementary to hybridize with their respective strands. A non-complementary nucleotide fragment may be attached to the 5′-end of the primer, with the remainder of the primer sequence being complementary to the diagnostic section of the target base sequence. Commonly, the primers are complementary except when non-complementary nucleotides may be present at a predetermined primer terminus as described.

As used herein, the terms “complementary” is used in reference to polynucleotides (i.e., a sequence of nucleotides) related by the base-pairing rules. For example, for the sequence “A-G-T,” is complementary to the sequence “T-C-A

The term “identical” means that two nucleic acid sequences have the same sequence or a complementary sequence.

The term “homologous” means that one single-stranded nucleic acid sequence may hybridize to a complementary single-stranded nucleic acid sequence. The degree of hybridization may depend on a number of factors including the amount of identity between the sequences and the hybridization conditions such as temperature and salt concentration. Preferably the region of identity is greater than about 5 bp, more preferably the region of identity is greater than 10 bp.

“Amplification” as used herein denotes the use of any amplification procedures to increase the concentration of a particular nucleic acid sequence within a mixture of nucleic acid sequences.

As used herein, the terms “restriction enzymes” and “restriction endonucleases” refer to bacterial enzymes, each of which cut double-stranded DNA at or near a specific nucleotide sequence. The specific nucleotide sequence is a “restriction site”.

The term “sample” as used herein is used in its broadest sense. A biological sample suspected of containing nucleic acid can comprise, but is not limited to, genomic DNA, cDNA (in solution or bound to a solid support), and the like.

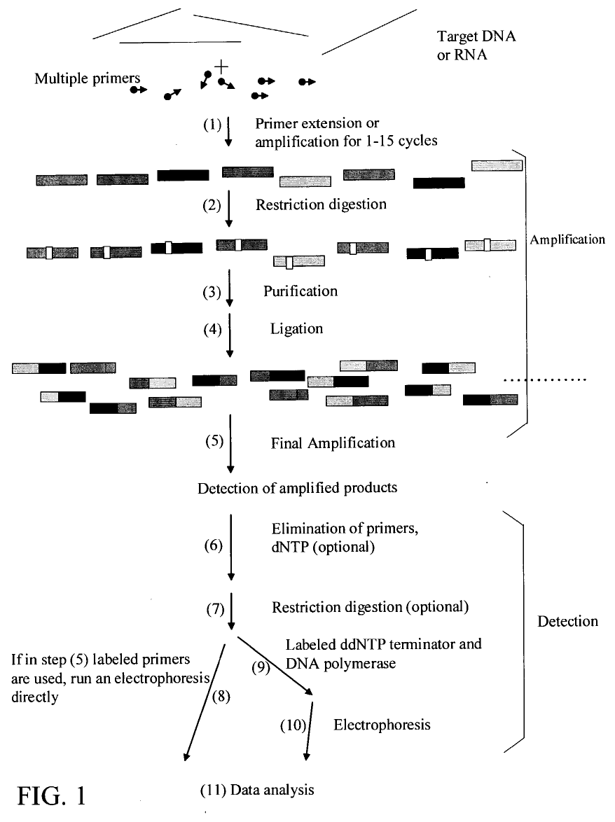

The present invention describes methods and compositions that allow the essentially simultaneous amplification and detection of a large number of different target nucleic acid sequences. The methods of invention comprise of amplification and detection processes as shown in FIG. 1.

A. Amplification

(1) Providing a set or sets of multiple primers with target nucleic acids in reactions of primer extension or amplification (also referred to as first amplification). Alternatively, providing different sets of multiple primers with target nucleic acids in separate reactions of primer extension or amplification. These reactions produce nucleic acid products in that each nucleic acid fragment comprises at least one restriction site. The reactions of primer extension or amplification are carried out at least once for 1-30 cycles, preferably 3-15 cycles. These reactions enable nucleic acid products derived from multiple targets to be created or amplified to a certain amount without introducing bias and non-specific products.

(2) Restriction digestion of products of step (1) on the restriction sites, preferably on the common first restriction sites, with cognate restriction enzyme. Optionally, before restriction digestion, the products may be immobilized to a solid support and purified.

(3) Purification of product of step (2) by any method known in the art.

(4) Join of product of step (3), preferably using a DNA ligase, whereby randomly joined nucleic acid fragments from said reaction or said separated reactions are created.

(5) Final amplification of joined products of step (4).

B. A Detection Method Using Electrophoresis

(6) Eliminating dNTP, primers and single stranded products from final amplification products. This step is optional. This elimination may be carried out by any digestion and/or purification method.

(7) Restriction digestion of products of step (6) on the restriction site, preferred on the second restriction sites, with cognate restriction enzymes.

(8) If fluorescence labeled primers are used in step (5), products may be directly subjected to a detection system, for example running an electrophoresis gel.

(9) If non-labeled primers are used in step (5), digested products are extended by a DNA polymerase in the presence of labeled terminator nucleotides. This step requires that in step (7) restriction digestion leaves digested DNA with 5′ protruding ends.

(10) Denaturing the extended double stranded products, running the DNA on gel or capillary electrophoresis.

(11) Analyzing data, preferably analyzing the data with aid of a computer software.

I. Materials

A. Target Sequences

The target sequence, which is the object of amplification and detection, can be any nucleic acid. The target sequence can be RNA, cDNA, genomic DNA, DNA contaminated by disease-causing microorganism and virus. The target sequence can also be DNA treated by chemical reagents, various enzymes and physical exposure. One example for detecting DNA methylation pattern is to treat DNA with methylation sensitive or resistant restriction endonuclease or treat DNA with sodium bisulfite which converts unmethylated cytosines to uracil.

B. Primers

Primers for use in the disclosed methods are oligonucleotides comprising 3′ sequences complementary to target sequences. This part of primer is referred to as complementary portion. The complementary portion of a primer can be any length that supports specific and stable hybridization between the primer and target sequence. Generally this is 9 to 40 nucleotides long, but is preferably 15 to 25 nucleotides long. It is preferred that complementary portion sequences of multiple primers used in reactions of primer extension or amplification are designed such that they have similar melting temperatures.

Primers also comprise additional sequences at the 5′ends of primers that are not complementary to target sequences. This sequence is referred to as non-complementary portion. The non-complementary portion of primer comprises sequence elements that are useful for various embodiments of the invention. Firstly, common restriction sites can be incorporated into the non-complementary portions of primers and are used for digesting and joining nucleic acid fragments amplified. Secondly, unique sequences or specific restriction sites in the non-complementary portions can serve as detection markers for distinguishing different alleles, different genes or any targets of interest. Thirdly, the non-complementary portion of primer can facilitate amplification. A primer having a sequence identical or homologous to the non-complementary portions of target specific primers in a set of primers is referred to as universal primer. The universal primer can be used in amplification after the target specific primers have been incorporated into an amplification product. Another advantage of using universal primer is that the universal primer can be the only primer that is labeled by expensive fluorescence dyes, capture moiety etc. which obviates the need to label every target-specific primers, thereby facilitating and simplifying detection and purification processes. For all theses purpose, a length of 10 to 50 nucleotides for the non-complementary portion is preferred, with the portion 15 to 35 nucleotides long being most preferred. The non-complementary portion can have any desired sequence. In general, the sequence of the non-complementary portions can be chosen such that it is not significantly similar to any sequence in target nucleic acids.

For each target sequence to be detected, there is generally a pair of target specific primers which comprises one reverse primer and one forward primer. If methods of the invention are used for amplification and detection of target sequences with mutations or SNPs and both alleles are desired to be detected in a reaction, two allele-specific forward primers along with a common non-discriminatory reverse primer can be included in a reaction. The allele-specific primers differ in their 3′ends which are complementary to either allele at a variable nucleotide site. Any additional mismatched nucleotide, which is known in the art to increase the specificity during polymerization, can be incorporated near 3′ end of allele specific primers (Gibbs et al, 1989).

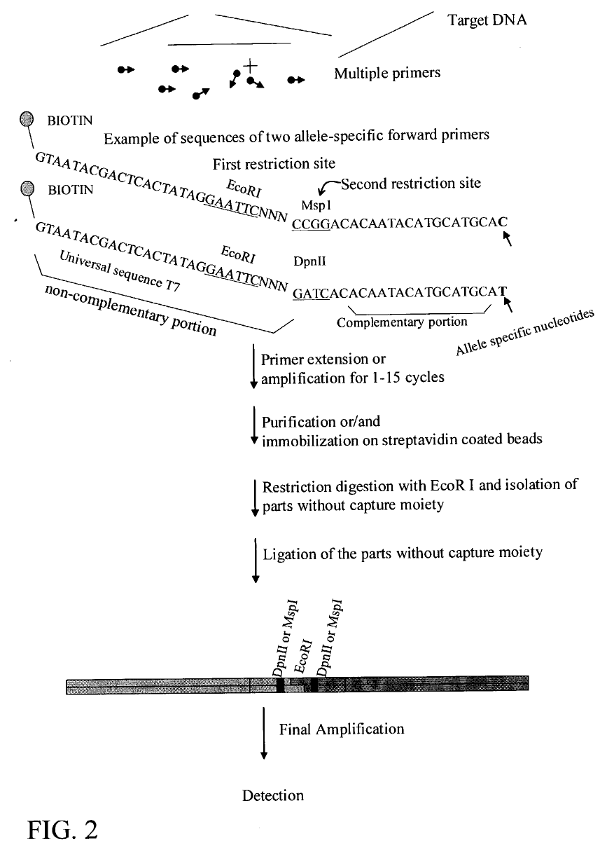

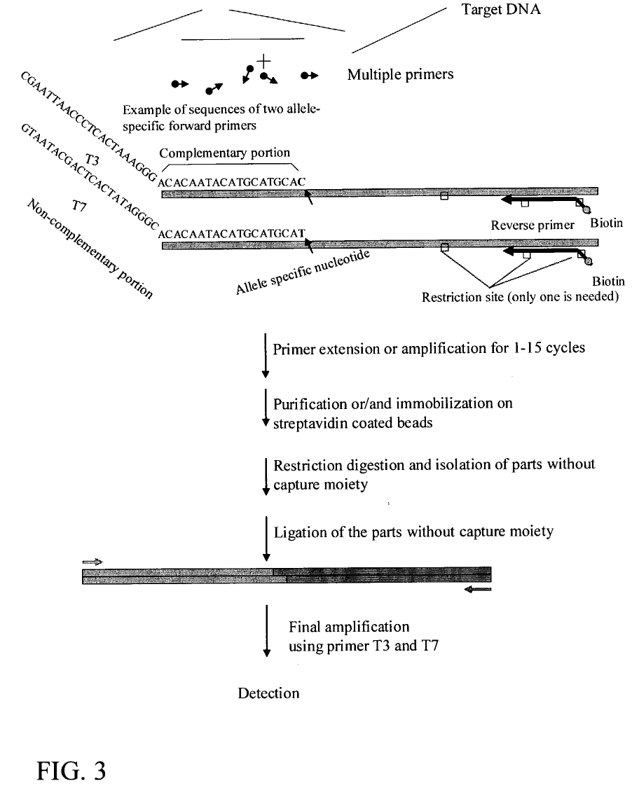

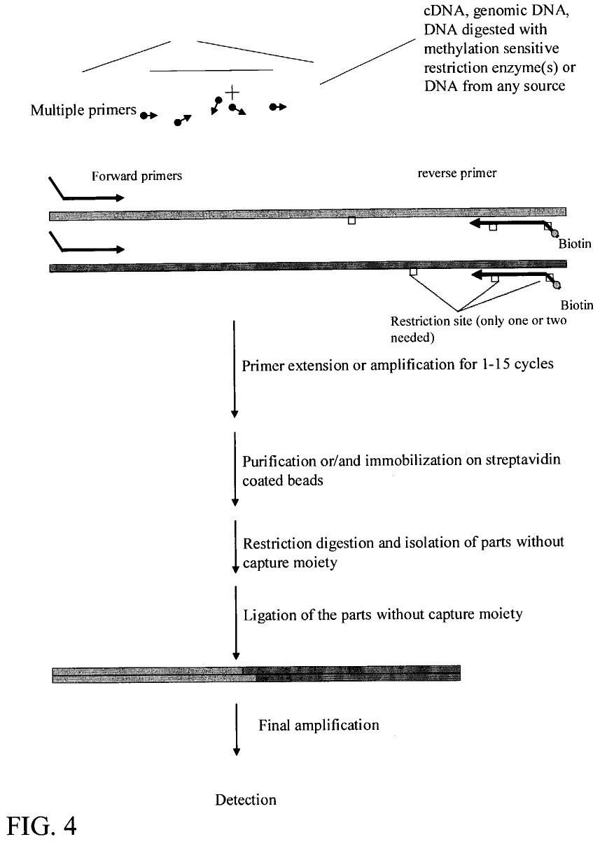

In most embodiments of the invention, primer sequences or target sequence to be amplified should comprise at least one restriction enzyme site. This restriction site can be the same restriction site for all targets. This common restriction site is referred to as first restriction site and is used for digesting and joining initial primer extension or amplification products. In some embodiments the first restriction site can be incorporated into either complementary portion or non-complementary portion of primers (FIG. 3 and FIG. 4), and preferably into non-complementary portion (FIG. 2).

The present invention also provides an oligonucleotide primer for the detection of a target nucleic acid sequence. The 5′ non-complementary portion of the primer comprises at least one restriction enzyme site, which acts as detection marker in a process of detecting the target nucleic acid sequence. A detection signal generated from enzymatic manipulation on the restriction site in a reaction product is indicative of the presence of the target nucleic acid sequence. The restriction site can be designed to be allele-specific, gene-specific or SNP-specific.

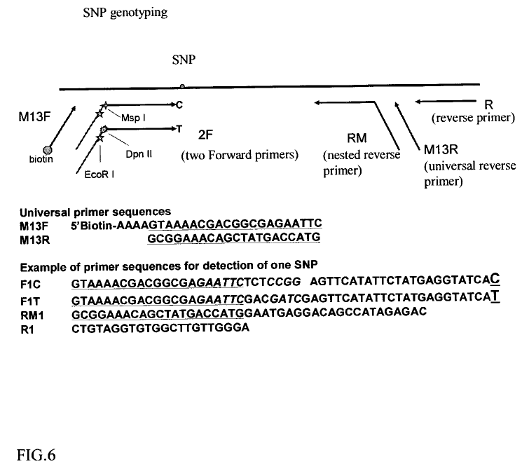

In one embodiment of the invention (FIG. 2 and FIG. 6), allele-specific restriction site is incorporated into 5′ region of or preferably immediately adjacent to complementary portion of allele-specific forward primer. If a common first restriction site is present, the allele-specific restriction site is referred to as second restriction site. The first restriction site is located 5′ of the second restriction site. A sequence linking the two restriction sites can be any sequence and can have any length. The allele-specific restriction site for a SNP may be designed such that one restriction site (for example Msp I) is incorporated into one allele-specific primer containing a 3′ end allelic nucleotide, whereas another restriction site (for example Dpn II) is incorporated into another allele-specific primer containing another 3′ end allelic nucleotide. In this way, the sequences of two allele-specific primers differ in their 3′ end allelic nucleotides and the second restriction sites. The second restriction sites are useful for detection of particular allele or SNPs. Alternatively, if the same second restriction enzyme site (for example Dpn II recognition sequence) is incorporated into the non-complementary portions of both allele-specific primers, the locations of second restriction sites can be different by several bases, preferably 1 to 9 bases. In this way, the two allele-specific primers differ in the locations of second restriction sites that result in length difference of final amplification products digested on the second restriction sites. In this embodiment, the non-complementary portions of two allele-specific primers for one target sequence have the same sequences except for the second restriction sites which are allele-specific.

In another embodiment of the invention, for genotyping and detecting multiple mutations or SNPs (FIG. 3), two different tailed allele-specific forward primers are used to analyze each variant. The complementary portions of the allele-specific forward primers have unique nucleotides at the 3′ ends or near 3′ends, which are complementary to the mutation or SNP nucleotides. The sequences of non-complementary portions of the two allele-specific forward primers are different, for example, one allele-specific primer having T7 promoter sequence and another allele-specific primer having T3 promoter sequence. The reverse primers comprise restriction enzyme sites either in the complementary portions or in the non-complementary portions, preferably in the non-complementary portions. Alternatively, if the primers do not comprise restriction sites, internal restriction sites on the target sequences can be used. It is preferred that these restriction sites are the same first restriction sites for all targets. It is further preferred that only one common restriction site is chosen or designed for each target.

In certain embodiments, primers can include one or more moieties incorporated into 5′ terminus or internally of primers that allow for the affinity separation of part of products associated with the label from unassociated part. Preferred capture moieties are those that can interact specifically with a cognate ligand. For example, capture moiety can include biotin, digoxigenin etc. Other examples of capture groups include ligands, receptors, antibodies, haptens, enzymes, chemical groups recognizable by antibodies or aptamers. The capture moieties can be immobilized on any desired substrate. Examples of desired substrates include, e.g., particles, beads, magnetic beads, optically trapped beads, microtiter plates, glass slides, papers, test strips, gels, other matrices, nitrocellulose, nylon. For example, when the capture moiety is biotin, the substrate can include streptavidin coated beads. Either reverse primers or forward primers in a set of multiple primers can be tagged by a capture moiety. More preferably, an universal primer having sequence identical or homologous to the non-complementary portion of either reverse primers or forward primers is tagged by a capture moiety

The term “a set of multiple primers” as used herein refers to a plurality of target specific primers and universal primers used in conjunction with each other, wherein each forward or reverse primers in the set has a functionally similar complementary portion and non-complementary portions, e.g., all of the complementary portions of primers have similar melting temperatures when hybridized to their targets; all of the non-complementary portions of forward primes or reverse primers have essentially the same sequence-specific hybridization properties to one or more universal primers and may comprise common restriction sites. However, the target complementary portion sequences and the allele-specific second restriction sites are different from one another in the set of multiple primers.

A set of multiple primers can include any desired number of target specific primers. It is preferred that a set of primers includes three or more primers. It is more preferred that a set of primers include 10 to 2000 primers. It is still more preferred that a set of primers include 30 to 800 primers. In general, the more primers used, the greater the level of amplification and detection that will be obtained. There is no fundamental upper limit to the number of primers that a set of primers can have. However, for a given detection system, the number of primers in a set of primers will generally be limited to the capacity of detection system. For example, if the detection system is a sequencing gel electrophoresis, it usually can separate 400 to 700 fragments. If the detection system is microarray, it can detect up to 10,000 target sequences.

In some embodiments of the invention, a set or several sets of nested primers are used in amplification. Nested primers for use in the amplification are oligonucleotides having sequence complementary to a region on a target sequence between reverse and forward primer targeting sites. The complementary portion of a nested primer can be any length that supports specific and stable hybridization between the primer and the target sequence. It is preferred that primers also contain additional sequence at the 5′ end of the nested primer that is not complementary to the target sequence (non-complementary portion).

If detection systems which measure molecular weight of amplified products such as electrophoresis are used, each primer pair in a set of multiple primers is designed so that each amplified product or the amplified product after digestion on restriction sites has distinct length for each target. In other words, the set of reverse primers, forward primers, nested primers and restriction sites are designed such that the amplified multiple products, when cut into fragments, are distinguishable by their sizes and/or labels. Fragment sizes may range from 20 to 2000 bases, allowing rapid detection by size on a number of known analytical systems. Detection of the fragment indicates the presence of the target sequence of interest. Preferably a fragment size ladder is included with the separation and detection of digested fragments to help identify the presence or absence of generated fragments. The fragments may be labeled by detection labels.

C. Detection Labels

To aid in detection and quantification of nuclei acids amplified using the disclosed methods, detection labels can be directly incorporated into amplified nucleic acids or can be extended by DNA polymerase on restriction digested products. As used herein, a detection label is any molecule that can be associated or added to amplified nucleic acid, directly or indirectly, and which results in a measurable, detectable signal, either directly or indirectly. Many such labels for incorporation into nucleic acids or coupling to nucleic acid probes are known to those of skill in the art. Examples of detection labels suitable for use in the disclosed method are radioactive isotopes, fluorescent molecules, phosphorescent molecules, enzymes, antibodies, and ligands.

In some embodiments of the invention, labeled nucleotide terminators are a preferred form of detection label since they can be directly incorporated into the digested amplification product in a polymerase extension. Another preferred form of detection label is labeled primer.

Methods for detecting and measuring signals generated by detection labels are also known to those of skill in the art. For example, the labeled fragments can be separated and detected by sequencing gel electrophoresis. The labeled fragments can be also detected in a microarray by hybridization.

D. Restriction Enzymes, DNA Polymerases and DNA Ligases

The disclosed methods make the use of restriction enzymes (also referred to as restriction endonucleases) for cleaving nucleic acids. Other nucleic acid cleaving reagents also can be used. Preferred nucleic acid cleaving reagents are those that cleave nuclei acid molecules in a sequence-specific manner.

Many restriction enzymes are known and can be used with the disclosed methods. Restriction enzymes generally have a recognition sequence and a cleavage site. Restriction enzyme digestion generates protruding ends or blunt ends at the cleavage site. For specific embodiments of the invention, restriction enzyme will cut amplified products at least once. The cutting sites are within a region between reverse and forward primer targeting sites, or are located on primer sequences. It is preferred that restriction enzymes generate 5′ protruding ends, if labeled nucleotide terminator and extension with a DNA polymerase are used in the detection process.

Any DNA polymerase can be used with the disclosed methods. If a thermo-cycle condition is required in the amplification, a thermostable DNA polymerase is preferred. In the detection process, for extending one or more labeled nucleotides both thermostable and non-thermostable DNA polymerase can be used. The preferred DNA polymerases are those routinely used in ordinary laboratories, for example, Taq DNA polymerase, Klenow fragment of DNA polymerase 1, Sequenase etc.

Suitable ligases used with the disclosed methods would include E. coli DNA ligase, T4 DNA ligase, Taq DNA ligase and AMPLIGASE®. T4 DNA ligase is the preferred ligase in most of embodiments. Most ligases require the presence of either ATP or NAD as an energy source. In addition, many ligases require a certain concentration of Mg⁺⁺.

II. Method

In a multiplex assay, it is desirable that quantitative measurements of different targets accurately reflect the true ratio of the target sequences. However, conventional multiplex PCR amplification methods inevitably introduce biases; the yields of final product do not proportionally represent the amount of target sequences in a sample. That is mainly because a relatively small difference in yield in one cycle of amplification results in a large difference in amplification yield after many cycles. The present invention is designed to overcome this limitation. It is based on the following principles. First, at least one restriction site is designed to be included in the initial primer extension or amplification products. Second, primer extension or amplification with a small number of cycles is carried out using a set of multiple primers that may anneal to multiple target sequences and prime amplification. It is desirable that this primer extension or amplification step yields a small amount of products from every target sequences, and either does not introduce any bias or minimizes the bias. It is further desirable that the cycle number of this initial amplification used is as low as possible, but high enough to ensure that it yields adequate products for next steps. Third, restriction digestion and ligation of the initial amplification products allow reorganization of nucleic acid fragments and create many new species of randomly joined nucleic acid products. The amplification of randomly joined nucleic acid products keeps the balance of overall yields of products from multiple original targets. Fourth, the amplified products may be re-cleaved by restriction enzymes, labeled and detected by various methods.

The present invention enables the multiple amplification and detection reactions to be used for the high throughput analysis of nucleic acid sequence. In these methods a number of genetic variants or expressed gene or any specific sequences may be quantitatively assayed. The detailed steps of the methods are as follows.

1. Provide a Set or Sets of Multiple Primers with Target Nucleic Acid in Reactions or Separate Reactions of Primer Extension or Amplification

The initial reactions can be either primer extension which is performed at least once, preferably more than once or amplification which is performed once or more than once each with a small number of cycles. Because the initial reactions is carried out with no cycles (primer extension) or with small number of cycles (amplification), a small amount of reaction products from every target sequences may be generated without introducing bias or with minimum bias.

In addition, the conditions of initial reactions also eliminate or minimize non-specific priming and amplification, whereby a better allelic differentiation can be achieved. In some embodiments of the invention, for amplification and detection of multiple mutations or SNPs, allele-specific primers are used to prime allele-specific extension. Mismatches at the 3′ end of a primer hinder extension of the primer during amplification. In conventional allele-specific PCR, mismatch discrimination was poor and was highly dependent on reaction conditions. The self-propagating nature of the mismatched extension in the conventional PCR has hindered development of robust high-throughput assays, and multiplexing of the reaction has been achieved only after extensive optimization of the reaction conditions (Ferrie et al. 1992). The present invention is designed to overcome the limitation of conventional PCR, whereby providing a robust high-throughput multiplex assays without the need of extensive optimization of the reaction conditions. The self-propagating nature of the mismatch extension which occurs in the conventional PCR is eliminated or minimized in the methods of the invention by carrying out initial allele-specific primer extension or amplification for a number of cycles which is kept as low as possible. Because of the low number of cycles, a primer extension product or an amplification product is created only when there is a perfect match between the allele-specific primers and a target sequence. The mismatched extension either does not occur or is not propagated, and a subsequent amplification does not inherit the mismatched extension.

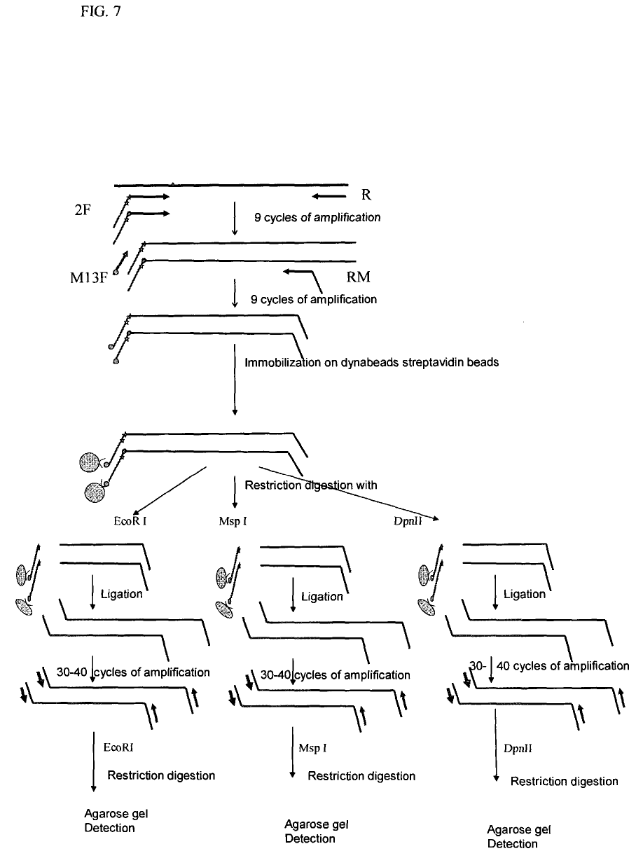

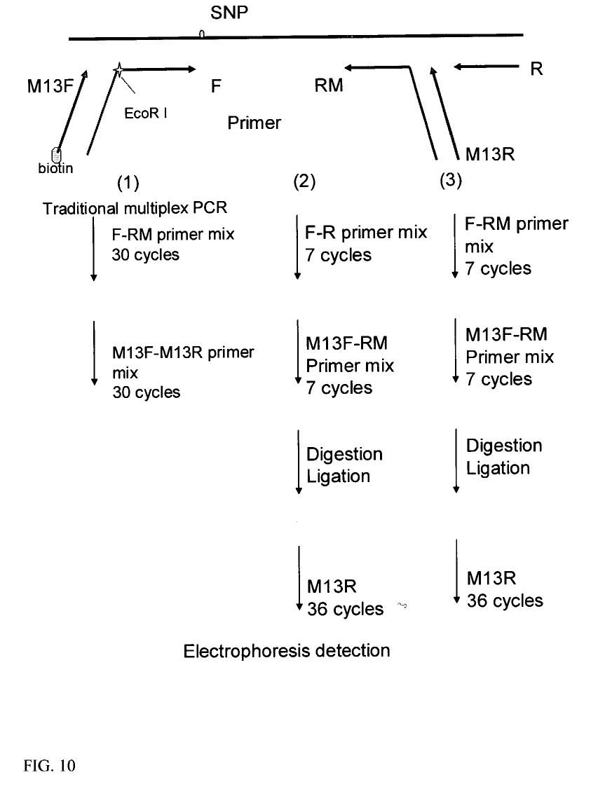

In some embodiments of the invention, forward primers or reverse primers or universal primers can comprises one or more capture moieties that permit affinity separation of the moiety-associated part from unassociated part of cleavage products. The primers can comprise, but not necessarily be limited to biotin, which permits affinity separation via binding to streptavidin attached to a solid support. For example, in FIG. 2, forward primers are tagged by biotin, whereas in FIGS. 3 and 4 reverse primers are tagged by biotin. In these examples of methods, every forward or reverse primer in a set of multiple primers is tagged by a capture moiety. Alternatively, the target specific primers in a set of multiple primers do not need to be tagged by capture moieties, but universal primers having sequences identical or homologous to the non-complementary portions of forward or reverse primers are tagged by capture moieties. The universal primers tagged by capture moieties are incorporated into the initial amplification products. Any means of incorporating universal primers into amplification products that is known to those skilled in the art can be used. For example, the universal primers can be mixed with the set of multiple primes in the first amplification step or can be added to the reaction at any cycle of the first amplification. Alternatively, or it is preferred that the methods described in FIGS. 6, 7, and 10 are used. The first amplification comprises two steps. In the first step, an amplification for a few cycles (9 cycles in FIGS. 7 and 7 cycles in FIG. 10) is carried out using a primer mixture of forward and reverse primer (F-R primer mix) or using a primer mixture of forward and nested reverse primer (F-RM). In the second step, after purification of the first step product, further amplification for 9 or 7 cycles is carried out using a primer mixture of forward universal primer and nested reverse primers (M13F-RM). This second step is used to incorporate universal primer with capture moiety.

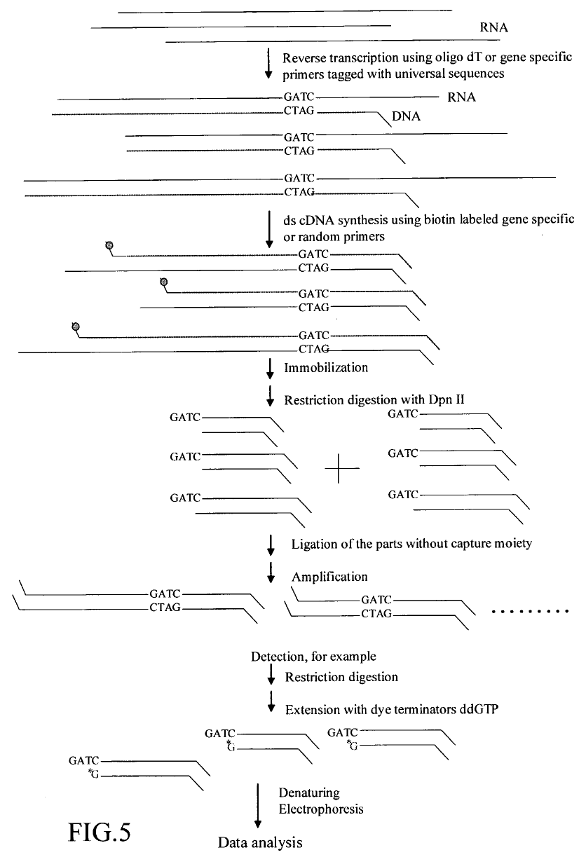

In yet another embodiment of the invention, the first amplification can be replaced by primer extensions. Primer extension can be performed either on DNA template or on RNA template. Here is an example using RNA template for first primer extension (FIG. 5). RNA targets are reverse transcribed into single stranded cDNAs with a set of target specific reverse primers, random primers, or oligo dT primers. The single stranded cDNAs are converted to double stranded cDNA with a set of target specific forward primer or random primers, which may be tagged by capture moieties. The double stranded cDNAs are subjected to the immediate next step of immobilization and restriction digestion. Alternatively, the double stranded cDNA may be amplified for a few cycles.

Thus, the initial reactions of primer extension or amplification are carried out once or more than once each for 1 to 30 cycles, or preferably 3 to 15 cycles. This initial primer extension or amplification reactions enable nucleic acid products derived from multiple targets to be created or amplified to a certain amount without introducing bias. It is preferred that the initial primer extension or amplification is carried out under standard conditions. It is more preferred that the initial primer extension or amplification is carried out under modified conditions. For example, a modified condition can be the use of modified buffer. Some modified buffers are commercially available, such as PCR buffers from Roche, Qiagen, Promega. Other modified conditions include, but not limited to, the use of low annealing temperature, long annealing time, and low concentration of each target specific primer in a set of multiple primers. In general, the low annealing temperature can be in a range from 5 degree C. to 20 degree C. lower than the actual Tm of complementary portions of target specific primers. The long annealing time can be more than 1 minute, preferably more than 2 minutes, most preferably more than 3 minutes. The low concentration of each target specific primer in a set of multiple primers is dependant on a certain circumstances of a particular experiment, generally the concentration of each primer in the initial reaction is lower than 50 nM, preferably lower than 10 nM, still preferably lower than 5 nM.

2. Purification, Immobilization, Restriction Digestion and Ligation

The products from primer extension or amplification can be purified by any method known in the art. Restriction digestion of these products is carried out by incubating DNA with appropriate restriction enzyme under optimal conditions. In preferred embodiments, wherein primers tagged with capture moieties that permit affinity separation are incorporated into the products, purifications before and after restriction digestion can be coupled with immobilization. If the capture moiety is biotin, the DNA products are immobilized via binding of the biotin to streptavidin which is attached to a solid support, one example of which is streptavidin coated beads. Following several washes, the immobilized DNA is incubated with appropriate restriction enzyme under optimal condition for the restriction digestion. The desired digested part, usually the supernatant DNA, is precipitated by any method know in the art. Usually, a carrier tRNA or glycogen is added to facilitate DNA precipitation. Ifjoining products from separate reactions is desired, before precipitation the digested products from separate reactions are mixed. The precipitated DNA is resolved in a ligation solution containing all necessary agents such as buffer, ATP (or NAD) and DNA ligase, the solution is incubated for several hours under optimal temperature for the ligase. Any DNA ligase can be used, T4 DNA ligase is a preferred enzyme.

In one embodiment of the invention, allele-specific forward primers comprise allele-specific restriction sites (the second restriction site), for example, Msp I sequence (CCGG) is associated with C allele; Dpn II sequence (GATC) is associated with T allele (FIG. 2, FIG. 6). Nucleic acid products from initial reactions of primer extension or amplification may be digested at the second restriction sites, and serve as ligation template for the ligation step. However, it is preferred that the common first restriction sites, herein for example EcoR I site (GAATTC) is incorporated into primers, are used for digesting and joining the initial amplification products.

3. Final Amplification

The final amplification is carried out using the ligated products as template for 2 to 50 cycles, more preferably 10 to 40 cycles. In this amplification the primers used may be target specific primes used in the first amplification. Alternatively, nested primers and preferably the nested primers tailed by universal sequences can be used. Using nested primers may eliminate nonspecific amplification. It is most preferred that the final amplification is carried out using one or more universal primers having sequences identical or homologous to the non-complementary portions of multiple target specific primers.

4. Detection

Following the final amplification, the amplified sequences can be detected and quantified using any of the conventional detection systems for nucleic acids such as detection of fluorescent labels, enzyme-linked detection systems, antibody-mediated label detection, and detection of radioactive labels. The high throughput microarray detection system may also be useful. It is preferred that systems that can separate and detect DNA fragments of different sizes are used. Since the amplified product is directly proportional to the amount of target sequence present in a sample, quantitative measurements reliably represent the amount of a target sequence in a sample.

One of preferred detection systems is electrophoresis, which may be gel or capillary electrophoresis and can be a DNA sequencer. Before loading to electrophoresis detection system, it is desirable that dNTP, primers and single stranded DNA from the final amplification products are eliminated. This elimination may be carried out by any method, one example of which is incubation with shrimp alkaline phosphatase and exonuclease I.

After eliminating dNTP, primers and single stranded DNA, and subsequent purification, the DNA products can be digested using first or second restriction enzymes. If both first and second restriction sites are incorporated into the amplification products, it is desirable that the amplification products are digested with second restriction enzymes on second restriction sites. After digestion, it is preferred that dye labeled ddNTP terminators are incorporated into the sticky ends of restriction digested fragments. This is achieved by one nucleotide extension in the presence of dye labeled nucleotide terminators and DNA polymerase. It is desirable that the labeled product is denatured before loading into electrophoresis system.

In one embodiment of the invention, to facilitate detection of allelic differences allele-specific forward primers comprise allele-specific restriction sites (the second restriction site), for example, Msp I sequence (CCGG) is associated with C allele; Dpn II sequence (GATC) is associated with T allele (FIG. 2, FIG. 6). The allele-specific restriction sites function as allele-specific markers in a detection process. In a preferred method for the detection process, the final amplified products are cleaved with two second restriction enzymes (Msp I and Dpn II in FIG. 2, FIG. 6, FIG. 7 and FIG. 9) on the second restriction sites. The 3′ ends of digested products are extended by a DNA polymerase in the presence of at least two different dye labeled nucleotide terminators, herein for example green dye labeled ddCTP and red dye labeled ddGTP. Thus, the C allele product if present is labeled in green, whereas the T allele if present is labeled in red. Following a gel or capillary electrophoresis, two alleles are distinguished by the different fluorescence labels. It is also possible that the same restriction sequences, for example Dpn II site, are incorporated into the second restriction sites for both allele-specific forward primers, but their locations are shifted by several bases, preferably 1 to 9 bases. In this way, in the detection process the final amplification products are cleaved with the second restriction enzyme Dpn II. The 3′ ends of digested products are extended by a DNA polymerase in the presence of at least one dye labeled nucleotide terminators, herein for example green ddCTP or red ddGTP. Thus, both C allele and T allele products (if both are present) are labeled with the same fluorescence either green or red. Following electrophoresis, two alleles are distinguished by different sizes of products.

The digested products produce a range of fragment sizes. The presence of any fragment of a particular length will indicate that a target sequence (a mutation, SNP, a gene etc) is present in a sample, since the size of any one fragment is unique to one specific target. The intensity of signal of any fragment of a particular length will indicate the amount of target sequence present in a sample.

Apart from electrophoresis detection systems, a number of other systems are available for the separation and detection of DNA fragments of different sizes. For example, high performance liquid chromatography and mass spectrometry are two known methods to separate compounds of differing lengths by size.

Apart from using dye labeled terminator in detection fragments, the detection fragments may be directly stained or labeled with radioactive labels, antibodies, luminescent dyes, fluorescent dyes, or enzyme reagents. Fluorescent dye is preferred. The detection fragment may be labeled by the fluorescent dye by using a direct DNA stain, by incorporation of a labeled nucleotide into the DNA during synthesis of the amplified DNA, or by using a labeled primer. Preferably the fluorescent dye label has an excitation and emission wavelength such that the dye may be excited at one wavelength and detected at a second wavelength. In addition, the dye should be detectable in the presence of other dyes.

Unless defined otherwise, all technical and scientific terms used herein have the same meanings as commonly understood by one of skill in the art to which the disclosed invention belongs. Although any methods and materials similar or equivalent to those described herein can be used in the practice or testing of the present invention, the preferred methods, devices, and materials are as described. All publication cited herein are hereby incorporated by reference.

Those skilled in the art will recognize, or be able to ascertain using no more than routine experimentation, many equivalents to the specific embodiments of the invention described herein.

### EXAMPLE 1

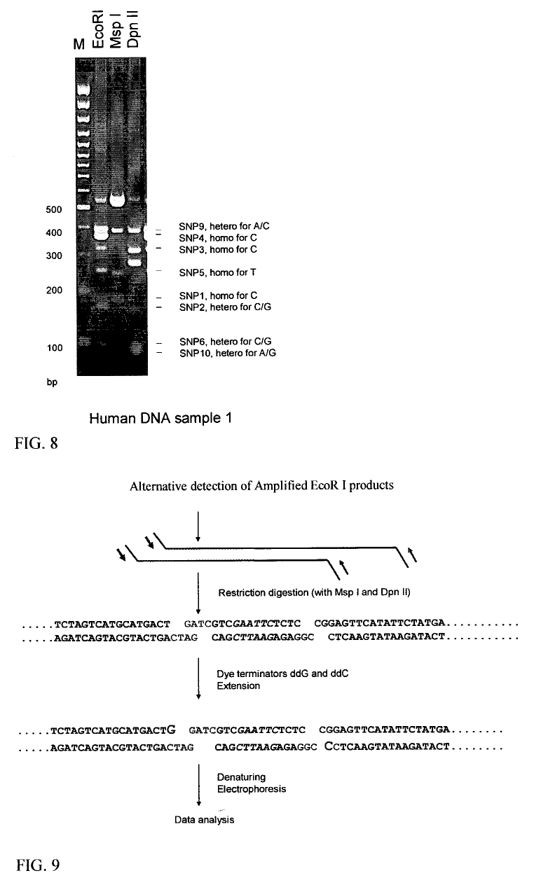

Multiplex Detection of SNPs (FIG. 6, FIG. 7 and FIG. 8)

All primers used in the subsequent experiments were synthesised by GENSET Singapore Biotech. Pte Ltd. Two universal primers M13F and M13R were designed, M13F was tagged with biotin. Two allele-specific forward primers were designed for each target. The forward primers comprise 3′ end complementary portion and 5′ end non-complementary portions which comprise a common first restriction site (EcoRI) and allele-specific second restriction sites (DpnII and Msp I) that are specific for each allele. Two reverse primers were designed for each target; the nested reverse primers comprise complementary portion and non-complementary portion.

TABLE 1

SEG ID NO 1-42

| | |
|---|---|
| PRIMER CODE | SEQUENCE 5′ - 3′ |

| | |
|---|---|
| M13F | 5′ Biotin-AAAAGTAAAACGACGGCGAGAATTC |
| M13R | CCGGAAACAGCTATGACCATG |
| F1C | GTAAAACGACGGCGAGAATTCTCTCCGGAGTTCATATTCTATGAGGTATCAC |
| F1T | GTAAAACGACGGCGAGAATTCGACGATCGAGTTCATATTCTATGAGGTATCAT |
| RM1 | GCGGAAACAGCTATCACCATGGAATGAGGACACCCATAGAGAC |
| R1 | CTGTAGGTGTGGCTTGTTGGGA |
| F2G | GTAAAACGACGGCGAGAATTCTCTCCCGCTGAAAGCAAGACTCAGAGG |
| F2C | GTAAAACGACGGCGAGAATTCGACGATCGCTGAAAGGAAGACTCAGAGC |
| RM2 | GCGGAAACAGCTATGACCATGCCTGGACAGTTACTCACAG |
| R2 | CCTGATTAGCACCCCAAGTC |
| F3T | GTAAAACGACGGCGAGAATTCTCTCCGGACTTCTGGTTTGCTCTTT |
| F3C | GTAAAACGACGGCGAGAATTCGACGATCGGACTTCTGGTTTCCTCTTC |
| RM3 | GCGGAAACAGCTATGACCATGCAGAGCTCAGGAGGAGTTAATG |
| R3 | ATAAATGTCACTGTTAGAGCCATCAA |
| F4A | GTAAACGACCGCCAGAATTCTCTCCGGCATTGGAGAACACCCAAGCAA |
| F4C | GTAAAACCACGGCGAGAATTCGACGATCATTGGAAGAACACCCAAGCAC |
| RM4 | GCCGAAACAGCTATGACCATGGACAGCCTGTCCACTCATCC |
| R4 | CTTGCATCACTGAGTCCCTC |
| F5T | CTAAAACCACGGCGAGAATTCTCTCCGGCTGAGGCAAACTTGAGGTTCT |
| F5C | GTAAAACCACGGCCAGAATTCGACGATCTCAGGCAAACTTGAGGTTCC |
| RM5 | GCCGAAACAGCTATGACCATGGGAAATGCTTTGTCCTTCCGTA |
| R5 | AGGGCCCACACCTCTGCCTT |
| F6C | GTAAAACGACGGCGACAATTCTCTCCGGTGTTATTTCTATCTCATTTCTTGAAC |
| F6C | GTAAAACGACCCCCAGAATTCGACGATCGTGTTATTTCTATCTGATTTCTTGAAG |
| RM6 | GCCGAAACAGCTATGACCATGGCTAGCCTCTCTTACCATAAC |
| R6 | CAATGCCCTAATCTCTTTGCCTT |
| F7T | GTAAAACGACGGCCACAATTCTCTCCGGCCAGGTGTCACTCAACATTGTAT |
| F7C | GTAAAACCACCCCGAGAATTCGACGATCCAGGTCTCACTGAAGATTGTAC |
| RM7 | CCCCAAACAGCTATGACCATGCTTTTCCCTCCTCCTAACTGC |
| R7 | GTCTTCCCTGACTCACCACTG |
| F8C | GTAAAACGACGGCGAGAATTCTCTCCGGCATAATCACAATGCTATTATTATGC |
| F8T | GTAAAACGACGGCCACAATTCCATGATCACAATGCTATTATTATGT |
| RM8 | GCGGAAACACCTATGACCATGATTCTTTGAAGATTAGACGCATC |
| R8 | CTTTCTCTATCTGTTCCACTAACCTAT |
| F9A | CTAAAACGACGGCGAGAATTCTCTCCCGTAACATACAACTGAAGCCA |
| F9C | GTAAAACGACGGCGAGAATTCGACGATCGTAACATACAACTGAAGCCC |
| RM9 | GCGGAAACACCTATGACCATGCAGTTTGTCCTCATCCTACTTG |
| R9 | CAGGCACCCTCTTTCCACATGA |
| F10A | CTAAAACGACGGCGAGAATTCTCTCCGGACTAGCACATTCACGGTTGAA |
| F10G | GTAAAACGACGGCGAGAATTCGACGATCGACTAGCAGATTCACGGTTGAG |
| RM10 | GCGGAAACAGCTATGACCATGGAAGAAGAAAGGCTGATGGC |
| R10 | GCAACTCATCTTTGATGGGTCATG |

Human genomic DNA samples were prepared by standard extraction from blood cells using the method of Kunkel L. M., Smith K. D., and Boyer S. H., 1977, Proc. Natl. Acad. Sci. USA, 74, 1245-49. Primers were diluted and final concentration of primers are as follows: M13R, 5.mu.M; M13F, 5.mu.M; Each of Forward (F) primers, 30 .mu.M; Each of reverse M (RM) primers, 30 .mu.M. Primer mix F-RM was made by mixing equal amount of each F primers and RM primers. Primer mix RM was made by mixing equal amount of each RM primers. Singleplex PCR were performed on each pair of primer which showed that the primer pairs for SNP 7 and 8 failed.

First Amplifications:

(1) Perform amplification using the following ingredients and conditions: 10×PCR Buffer 5 .mu.l, 10 mM dNTPs 1-2.5 .mu.l, F-RM primer 6 .mu.l, Water 34 .mu.l, Taq polymerase (5 U/.mu.l) 1 .mu.l, Human genomic DNA 2 .mu.l (100 ng-500 ng). 10×PCR buffer used are either standard buffer containing 500 mM KCl, 100 mM Tris-HCl pH 8.3, 15 mM MgCl.sub.2, 1% gelatin, or buffers accompanied with kits provided by manufactures Promega or Roche (Expand long PCR kit). Reactions were carried out at 94 C for 1 min; 2 cycles of 30 sec at 93 C, 9 sec at 60 C, 3 min at 51 C, 1 min at 50 C, 1 min at 49 C, 2 min at 66 C, and 5 cycles of 30 sec at 93 C, 9 sec at 66 C, 3 min at 51 C, 1 min at 50 C, 1 min at 49 C, 2 min at 66 C, followed by a final extension step at 68 C for 6 min. Reactions were also carried out using a simple cycle condition which gave similar result: 94 C for 1 min; 7-9 cycles of 30 sec at 93 C, 3 min at 51 C, 2 min at 68 C, followed by a final extension step at 68 C for 6 min.

(2) Purify the above product using Qiagen PCR purification kit according the manufacture's protocol. Elute the DNA in 40 .mu.l of elution buffer.

(3) Perform reaction using the following ingredients and conditions: 10×PCR Buffer 5 .mu.l, 10 mM dNTPs 1-2.5 .mu.l, RM primer 3 .mu.l, M13F primer 3 .mu.l, Taq polymerase (5 U/.mu.l) 1 .mu.l, Eluted DNA 36 .mu.l. Reactions were carried out at 94 C for 1 min; 7-9 cycles of 3 min at 51 C, 2 min at 68 C, followed by a final extension step at 68 C for 6 min.

(4) Binding biotinylated DNA onto magnetic beads. Add 50 .mu.l Dynabead M-28-Steptavidin slurry to a tube. Use magnet to immobilize beads and remove supernatant. Wash beads once. Add 100 .mu.l 2×B+W to the tube, 50 .mu.l water and 50 .mu.l amplification products to the tube. Incubate 15 minutes at room temperature. Mix intermittently. Wash three times, removing the wash each time. Wash twice with 1× restriction enzyme buffer. Resuspend beads in 150 .mu.l 1× restriction enzyme buffer. Proceed immediately to step 5 below.

(5) Restriction digestion, precipitation, and ligation. Divide beads into three parts, each part has 50 .mu.l of beads. Add 2 mu.l EcoR I (20 units), or 2 mu.l Msp 1 (20 units) or 2 .mu.l Dpn II (20 units) into each part of the beads. Incubate at 37 degree C. for 2 hours. Gently mixing intermittently. Magnet, collect supernatant. Heat to inactivate restriction enzymes at 66 degree C. for 15 min. Ethanol precipitate: mix 50 .mu.l sample, 1 .mu.l glycogen, 30 .mu.l NH4OAC (7.5M), 240 .mu.l ethanol. Spin for 30 min at 4 degree C. Wash once with 70% ethanol, centrifuge and remove ethanol. Resuspend DNA in 5 .mu.l of ligation mixture containing 0.5 .mu.l 10× ligation buffer, 0.5 .mu.l T4 DNA ligase, 4 .mu.l water. Incubate overnight at 16 degree C. Add 25 .mu.l of water to ligation mixture and proceed to amplification below, or store at −20 degree C.

Final Amplification

(6) Mix the following components in each of three tubes: 10×PCR Buffer 5 .mu.l, 10 mM dNTPs 1-2.5 .mu.l, M13R primer 9 .mu.l, Ligated DNA 15 .mu.l, Taq polymerase (5 U/.mu.l) 1 mu.l, Water 27 .mu.l. Reactions were carried out at 94 C for 1 min; 2 cycles of 20 sec at 93 C, 45 sec at 54 C, 1 min at 68 C; 2 cycles of 20 sec at 93 C, 45 sec at 52 C, 1 min at 68 C; 36 cycles of 20 sec at 93 C, 45 sec at 50 C, 1 min at 68 C, followed by a final extension step at 68 C for 6 min.

(7) Restriction digestion. Take 18 .mu.l of amplification product from each of three tubes, add 1.5 .mu.l restriction buffer, add 2 .mu.l EcoR I, 2 .mu.l Msp I, 2 .mu.l Dpn II to corresponding tubes with an appropriate restriction digestion in step 5. Incubate at 37 degree C. for 2 hours. Electrophoresis separates the digestion products.

Result: The result is shown in FIG. 8. All working primer pairs for 8 SNPs give clear either heterozygous or homozygous patterns. The EcoR I lane reveals all 8 fragments; the Msp I lane reveals fragments corresponding alleles that are produced with Msp I tagged allele-specific primers; the Dpn II lane reveals fragments corresponding other alleles that are produced with Dpn II I tagged allele-specific primers. The SNP 4 in lane Dpn II has a fragment shorter than its corresponding fragment in lane EcoR I, because of internal Dpn II site in the amplified fragment.

### EXAMPLE 2

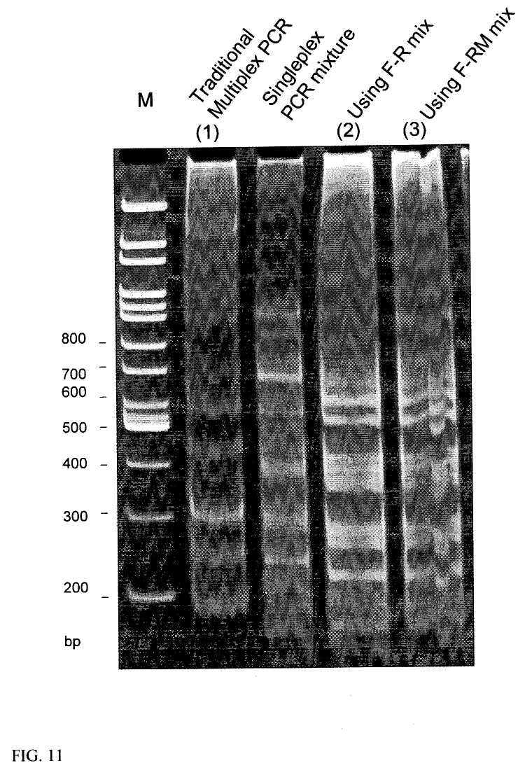

Multiplex Amplification of 70 Fragments Comprising SNPs (FIG. 10 and FIG. 11)

All primers used in the subsequent experiments were synthesised by GENSET Singapore Biotech. Pte Ltd. Two universal primers M13F and M13R were designed, M13F was tagged with biotin. One forward and two reverse primers were designed for each target. The forward primers comprise 3′ end complementary portion and 5′ end non-complementary portions which comprise a common first restriction site (EcoRI). Two reverse primers were designed for each target; the nested reverse primers comprise complementary portion and non-complementary portion (FIG. 10). The sequences of primers (209 primers) are listed in the sequence listing (SEQ ID NO 43-251). Primers were diluted as follows: M13R, 5 .mu.M; M13F, 5 .mu.M; Each of forward (F) primers, 30 .mu.M; Each of reverse M (RM) primers, 30 .mu.M; Each of reverse (R) primers, 30 .mu.M. Primer mix F-RM was made by mixing equal amount of each F primers and RM primers. Primer mix RM was made by mixing equal amount of each RM primers. Primer mix R was made by mixing equal amount of each R primers. Singleplex PCR were performed on each pair of primer which showed that 3 primer pairs were not working. All singleplex PCR products were mixed together to serve as a control in running gels.

First Amplifications

(1) Two amplification reactions were performed using the following ingredients: F-R reaction: 10×PCR Buffer 5 .mu.l, 10 mM dNTPs 1-2.5 .mu.l, F-R primer 6 .mu.l, H2O 34 .mu.l, Taq polymerase (5 U/.mu.l) 1 .mu.l, and Human genomic DNA 2 .mu.l (100 ng-500 ng). F-RM reaction: 10×PCR Buffer 5 .mu.l, 10 mM dNTPs 1-2.5 .mu.l, F-RM primer 6 .mu.l, H2O 34 .mu.l, Taq polymerase (5 U/.mu.l), 1 .mu.l, and Human genomic DNA, 2 .mu.l (100 ng-500 ng). 10×PCR buffer used are either standard buffer containing 500 mM KCl, 100 mM Tris-HCl pH 8.3, 15 mM MgCl.sub.2, 1% gelatin, or buffers accompanied with kits provided by manufactures Promega or Roche (Expand long PCR kit). Reactions were carried out at 94 C for 1 min; 7 cycles of 30 sec at 93 C, 3 min at 51 C, 2 min at 68 C, followed by a final extension step at 68 C for 6 min. Purify the amplification products using Qiagen PCR purification kit according the manufacture's protocol. Elute the DNA in 40 .mu.l of elution buffer.

(2) Perform two reactions using the same following ingredients: 10×PCR Buffer 5 .mu.l, 10 mM dNTPs 1-2.5 .mu.l, RM primer 3 .mu.l, M13F primer 3 .mu.l, Taq polymerase (5 U/.mu.l) 1 .mu.l, and Eluted DNA 36 .mu.l, Reactions were carried out at 94 C for 1 min; 7 cycles of 30 sec at 93 C, 3 min at 51 C, 2 min at 68 C, followed by a final extension step at 68 C for 6 min.

(3) Binding biotinylated DNA onto magnetic beads. Add 50 .mu.l Dynabead M-28-Steptavidin slurry to each of two tubes. Use magnet to immobilize beads and remove supernatant. Wash beads once. Add 100 .mu.l 2×B+W to the tube, 50 .mu.l water and 50 .mu.l amplification products to each tube. Incubate 15 minutes at room temperature. Mix intermittently. Wash three times, removing the wash each time. Wash twice with 1× restriction enzyme buffer. Resuspend beads in 50 .mu.l 1× restriction enzyme buffer. Proceed immediately to step 5 below.

(4) Restriction digestion, precipitation and ligation. Add 2 .mu.l EcoR I (20 units) into each beads of two reaction tubes. Incubate at 37 degree C. for 2 hours. Gently mixing intermittently. Magnet, collect supernatant. Heat to inactivate restriction enzymes at 66 degree C. for 15 min. Ethanol precipitation: mix 50 .mu.l sample, 1 .mu.l glycogen, 30 .mu.l NH4OAC (7.5M), and 240 .mu.l ethanol. Spin for 30 min at 4 degree C. Wash once with 70% ethanol, centrifuge and remove ethanol. Resuspend DNA in 5 .mu.l of ligation mixture containing 0.5 .mu.l 10× ligation buffer, 0.5 .mu.l T4 DNA ligase, 4 .mu.l water. Incubate overnight at 16 degree C. Add 25 .mu.l of water to ligation mixture and proceed to amplification below, or store at −20 degree C.

Final Amplification

(5) Mix the following components in each of two reaction tubes. 10×PCR Buffer 5 .mu.l, 10 mM dNTPs 1-2.5 .mu.l, M13R primer 9 .mu.l, Ligated DNA 15 .mu.l, Taq polymerase (5 U/.mu.l) 1 .mu.l, and Water 27 .mu.l. Reactions were carried out at 94 C for 1 min; 2 cycles of 20 sec at 93 C, 45 sec at 54 C, 1 min at 68 C; 2 cycles of 20 sec at 93 C, 45 sec at 52 C, 1 min at 68 C; 36 cycles of 20 sec at 93 C, 45 sec at 50 C, 1 min at 68 C, followed by a final extension step at 68 C for 6 min. Take 18 .mu.l of amplification product from each of two reactions, add 1.5 .mu.l restriction buffer, add 2 .mu.l EcoR I to each reaction. Incubate at 37 degree C. for 2 hours. Electrophoresis separates the digestion products.

A control conventional multiplex PCR was performed as follows: First amplification, 10×PCR Buffer 5 .mu.l, 10 mM dNTPs 1-2.5 .mu.l, F-RM primer 6.mu.l, Water 34 .mu.l, Taq polymerase (5 U/.mu.l) 1 .mu.l, and Human genomic DNA 1 .mu.l (100 ng-500 ng). Reactions were carried out at 94 C for 1 min; 30 cycles of 30 sec at 93 C, 2 min at 51 C, 2 min at 68 C, followed by a final extension step at 68 C for 6 min. Second amplification was performed using the following ingredients: 10×PCR Buffer 5 .mu.l, 10 mM dNTPs 1-2.5 .mu.l, M13R primer 3 .mu.l. M13F primer 3 .mu.l, Taq polymerase (5 U/.mu.l) 1 .mu.l, First amplification product 2 mu.l, and water 35 mu.l. Reactions were carried out at 94 C for 1 min; 30 cycles of 30 sec at 93 C, 1 min at 51 C, 1 min at 68 C, followed by a final extension step at 68 C for 6 min.

Result. An acrylamide gel was run with loading products from traditional multiplex PCR, singleplex PCR mixture, F-R reaction and F-RM reaction. The result is shown in FIG. 11. The result demonstrates that the traditional PCR gave a poor result compared with singleplex PCR mixture control. The results from F-R and F-RM reactions gave similar patterns and are comparable with the singleplex PCR mixture control, indicating most of target fragments were amplified. The result also demonstrates that there is no much difference between reaction F-R and reaction F-RM, indicating that the use of nested reverse primers does not offer significant advantage in this experiment.

### EXAMPLE 3

Multiplex Detection of Enteric Bacteria

Two universal primers M14F and M14R were designed, M14F was tagged with biotin. One forward and two reverse primers were designed for each target. The forward primers comprise 3′ end complementary portion and 5′ end non-complementary portions which comprise a common first restriction site (EcoRI). Two reverse primers were designed, one of which the nested reverse primers comprise complementary portion and non-complementary portion. Primers were designed to detect Vibrio cholerae (target genes cholera toxin ctx and rtx), Salmonella typhi (Vi antigen B,ViaB), Shigella dysenteriae (O-antigen polymerase, Rfc), Salmonella species (Invasion protein InvA), Shigella species (Invasive protective antigen H), E. coli O157 (Intimin and rfbA), and Listeria monocytogenes (hly and inlB).

TABLE 2

SEQ ID NO 252-283

| | |
|---|---|
| PRIMER CODE | SEQUENCE 5′ - 3′ |

| | |
|---|---|
| M14F | 5′-Biotin-AAAAGTGGAACGACGGCGAGAATT |
| M14R | GCGGAAACAGCTATGACCATG |
| FCTX | GTGGAACGACGGCGAGAATTCGGGGCATACAGTCCTCATCCA |
| RMCTX | GCGGAAACAGCTATGACCATGGGAAACCTGCCAATCCATAAC |
| RCTX | GTGGAACGACGGCGAGACTCTTCCCTCCAAGCTCTATGCTC |
| FRTX | GTGGAACGACGGCGAGAATTCGACGAAGATCATTGACGACCTC |
| RMRTX | GCGGAAACAGCTATGACCATGCCGCTTCATCGTCGTTATGTG |
| RRTX | GTGGAACGACGGCGAGAGATAGGTGGTGTGATGCTGCTCAC |
| FVIAB | GTGGAACGACGGCGAGAATTCCCTTGCACGTTTTTGGTTGACAGA |
| RMVIAB | GCGGAAACAGCTATGACCATGGATAGCGCGGCTCACGTACTC |
| RVIAB | GTGGAACGACGGCGAGACTGAATCCGGCAATAACAGATAGC |
| FINVA | GTGGAACGACGGCGAGAATTCGGTGAAATTATCGCCACGTTCG |
| RMINVA | GCGGAAACAGCTATGACCATGCACCGTCAAAGGAACCGTAAAG |
| RINVA | GTGGAACGACGGCGAGGGTCATCCCCACCGAAATACC |
| FRFC | GTGGAACGACGGCGAGAATTCGACTGATACCATGGTGCAAAAGC |
| RMRFC | GCGCAAACAGCTATGACCATGCTCGGGATTGGCAGCCTTTAATC |
| RRFC | GTGGAACGACGGCGAGAGGGTAAGTTCTCTTCAGACCCTGAACG |
| FIPAH | GTGGAACGACGGCGAGAATTCACGGCTTCTGACCATAGCTTCGGCAGTG |
| RMIPAH | GCGGAAACAGCTATGACCATGCGATAATGATACCGGCGCTCTG |
| RIPAH | GTGGAACGACGGCCAGATCCTGGTCCATCAGGCATCAGAAG |
| F157 | CTCGAACGACGGCGAGAATTCGGCTTCAAGATCTTTGGCAAC |
| RM157 | CCGGAAACAGCTATGACCATGCAGTTTGTCGAAATCGCAGCAG |
| R157 | GTGGAACGACGGCGAGAGCAACAGGAGTCCAATACTCAGTC |
| FEAE | GTGGAACGACCCCGAGAATTCACCCCTTACGATCTGGTTCAGC |
| RMEAE | GCGGAAACAGCTATGACCATGCCTGGTAGTCTTGTGCGCTTTG |
| REAE | GTGGAACGACGGCGAGACTGCCACCTTGCACATAAGCAG |
| FHLY | GTGGAACGACGGCGAGAATTCTACCAATTGCGCAACAAACTGAAG |
| RMHLY | GCGGAAACAGCTATGACCATGGCTCGAAATTGCATTCACAACTTG |
| RHLY | GTGGAACGACGGCGAGATTAGTCATTCCTGGCAAATCAATGC |
| FINLB | GTGGAACGACGGCGAGAATTCGTGATGATGGCGATTATGAAAAACC |
| RMINLB | GCGGAAACAGCTATGACCATGCTCTTTCAGTGGTTGGGTTACTCTC |
| RINLB | GTGGAACCACGGCGAGACCGTTCCATCAACATCATAACTTACTG |

Bacteria DNA samples were prepared by standard extraction from culture using a kit provided by Promega. Each bacterium DNA was prepared at a concentration of 1 ng/.mu.l. DNA mixture was prepared by mixing equal amount of each bacterium DNA. Primers were diluted as follows: M14R, 5 .mu.M; M14F, 5 .mu.M; Each of Forward (F) primers, 20 .mu.M; Each of reverse M (RM) primers, 20 .mu.M; Each of reverse (R) primers, 20 .mu.M. Primer mix F-RM was made by mixing all F primers and all RM primers. Primer mix RM was made by mixing all RM primers. Primer mix R was made by mixing all R primers. Primer mix F-R was made by mixing all F primers and all R primers. The above primer mixtures were diluted to a concentration of 0.1 .mu.M for each individual primer. Singleplex PCR were performed on each pair of primer which showed that all primer pairs worked. All singleplex PCR products were mixed together to serve as a control in gel electrophoresis.

Experiments were performed to examine three conditions: (a) procedure using nested reverse primer and biotin mediated immobilization; (b) procedure using nested reverse primer without biotin mediated immobilization; and (c) procedure not using nested reverse primer, nor biotin mediated immobilization. (a) First amplification was performed using the following ingredients: 10×PCR Buffer 5 .mu.l, MgCI2 3 .mu.l. 10 mM dNTPs 1 .mu.l, F-R primer 2 .mu.l, Water 28 .mu.l, Taq polymerase (5 U/.mu.l) 1 .mu.l, and Bacteria DNA mixture 10 .mu.l. Reactions were carried out at 94 C for 1 min; 7 cycles of 30 sec at 94 C, 3 min at 52 C increasing to 70 C in 5 min, 9 sec at 70 C, followed by a final extension step at 70 C for 6 min. Purify the above products using Qiagen PCR purification kit according the manufacture's protocol. Elute the DNA in 40 .mu.l of elution buffer. Perform another amplification reaction using the following ingredients: 10×PCR Buffer 5 .mu.l, MgCl2 3 .mu.l, 10 mM dNTPs 1 .mu.l, RM primer 3 .mu.l, M14F primer 6 .mu.l, Taq polymerase (5 U/.mu.l) 1 .mu.l, Eluted DNA 31 mu.l. Reactions were carried out at 94 C for 1 min; 7 cycles of 30 sec at 94 C, 3 min at 52 C increasing to 70 C in 5 min, 9 sec at 70 C, followed by a final extension step at 70 C for 6 min.

After binding biotinylated DNA onto magnetic beads and subsequent restriction digestion, precipitation, and ligation, final amplification was performed by mixing the following components. 10×PCR Buffer 5 .mu.l, MgCl2 3. mu.l, 10 mM dNTPs 1.mu.l, M14R primer 6 .mu.l, Ligated DNA 24 .mu.l, Taq polymerase (5 U/.mu.l) 1 .mu.l. Reactions were carried out at 94 C for 1 min; 2 cycles of 20 sec at 93 C, 45 sec at 54 C, 1 min at 68 C; 2 cycles of 20 sec at 93 C, 45 sec at 52 C, 1 min at 68 C; 36 cycles of 20 sec at 93 C, 45 sec at 51 C, 1 min at 68 C, followed by a final extension step at 68 C for 6 min. Restriction digestion for detection: Take 18 .mu.l of the amplification product, add 1.5 .mu.l restriction buffer, add 2 .mu.l EcoR I to the reaction. Incubate at 37 degree C. for 2 hours. Electrophoresis separates the digestion products.

A control traditional multiplex PCR were performed as follows: First amplification: 10×PCR Buffer 5 .mu.l, MgCl2 3.mu.l, 10 mM dNTPs 1 .mu.l, F-R primer 2 .mu.l, Water 28 .mu.l, Taq polymerase (5 U/.mu.l) 1 .mu.l, Bacteria DNA mixture 10 .mu.l. Reactions were carried out at 94 C for 1 min; 36 cycles of 30 sec at 94 C, 2 min at 52 C, 1 min at 70 C, followed by a final extension step at 70 C for 6 min. Second Amplification: 10×PCR Buffer 5 mu.l, MgCl2 3 .mu.l, 10 mM dNTPs 1 .mu.l, M14R primer 4 .mu.l, M14F primer 4 .mu.l, First amplification product 10 .mu.l, Taq polymerase (5 U/.mu.l) 1 mu.l, Water 22 .mu.l. Reactions were carried out at 94 C for 1 min; 2 cycles of 20 sec at 93 C, 45 sec at 60 C, 1 min at 70 C; 2 cycles of 20 sec at 93 C, 45 sec at 57 C, 1 min at 70 C; 36 cycles of 20 sec at 93 C, 45 sec at 54 C, 1 min at 70 C, followed by a final extension step at 68 C for 6 min.

(b) First amplification was performed using the following ingredients: 10×PCR Buffer 5 .mu.l, MgCI2 3.mu.1, 10 mM dNTPs 1 .mu.l, F-R primer 2 .mu.l, Water 28 .mu.l, Taq polymerase (5 U/.mu.l) 1 .mu.l, Bacteria DNA mixture 10 .mu.l. Reactions were carried out at 94 C for 1 min; 7 cycles of 30 sec at 94 C, 3 min at 52 C increasing to 70 C in 5 min, 9 sec at 70 C, followed by a final extension step at 70 C for 6 min. Purify the above products using Qiagen PCR purification kit according the manufacture's protocol. Elute the DNA in 40 .mu.l of elution buffer. Perform another reaction using the following ingredients: 10×PCR Buffer 5 .mu.l, MgCI2 3.mu.l, 10 mM dNTPs 1 .mu.l, RM primer 3 .mu.l, M14F primer 6 .mu.l, Taq polymerase (5 U/.mu.l) 1 .mu.l, Eluted DNA 31 .mu.l. Reactions were carried out at 94 C for 1 min; 7 cycles of 30 sec at 94 C, 3 min at 52 C increasing to 70 C in 5 min, 9 sec at 70 C, followed by a final extension step at 70 C for 6 min. Purify the above products using Qiagen PCR purification kit according the manufacture's protocol. Elute the DNA in 40 .mu.l of elution buffer.

Without immobilization, the above DNA products were restriction digested, ethanol precipitaed, and ligated with T4 DNA ligase. Final amplification was performed by mixing the following components: 10×PCR Buffer 5 .mu.l, MgCl2 3 .mu.l, 10 mM dNTPs 1 .mu.l, M14R primer 6 .mu.l, Ligated DNA 24 .mu.l, Taq polymerase (5 U/.mu.l) 1 .mu.l. Reactions were carried out at 94 C for 1 min; 2 cycles of 20 sec at 93 C, 45 sec at 54 C, 1 min at 68 C; 2 cycles of 20 sec at 93 C, 45 sec at 52 C, 1 min at 68 C; 36 cycles of 20 sec at 93 C, 45 sec at 51 C, 1 min at 68 C, followed by a final extension step at 68 C for 6 min. Restriction digestion: Take 18 .mu.l of the amplification product, add 1.5 .mu.l restriction buffer, add 2 .mu.l EcoR I to the reaction. Incubate at 37 degree C. for 2 hours. Electrophoresis separates the digestion products.

(c) Amplification was performed using the following ingredients: 10×PCR Buffer 5 .mu.l, MgCl2 3.mu.l, 10 mM dNTPs 1 .mu.l, F-RM primer 2 .mu.l, Water 28 .mu.l, Taq polymerase (5 U/.mu.l) 1 .mu.l, Bacteria DNA mixture 10 .mu.l. Reactions were carried out at 94 C for 1 min; 15 cycles of 30 sec at 94 C, 3 min at 52 C increasing to 70 C in 5 min, 9 sec at 70 C, followed by a final extension step at 70 C for 6 min. Purify the above products using Qiagen PCR purification kit according the manufacture's protocol. Elute the DNA in 40 .mu.l of elution buffer.

After restriction digestion, precipitation and ligation, final amplification was performed using the following components. 10×PCR Buffer 5 .mu.l, MgCl2 3. mu.l, 10 mM dNTPs 1.mu.l, M14R primer 6 .mu.l, Ligated DNA 24 .mu.l, Taq polymerase (5 U/.mu.l) 1 .mu.l. Reactions were carried out at 94 C for 1 min; 2 cycles of 20 sec at 93 C, 45 sec at 54 C, 1 min at 68 C; 2 cycles of 20 sec at 93 C, 45 sec at 52 C, 1 min at 68 C; 36 cycles of 20 sec at 93 C, 45 sec at 51 C, 1 min at 68 C, followed by a final extension step at 68 C for 6 min. Restriction digestion: Take 18 .mu.l of the amplification product, add 1.5 .mu.l restriction buffer, add 2 .mu.l EcoR I to the reaction. Incubate at 37 degree C. for 2 hours. Electrophoresis separates the digestion products.

Result: The results show that all three conditions tested gave comparable results which clearly demonstrated an improved amplification compared with traditional multiplex PCR. The condition (a) using nested primers and biotin-beads mediated immobilization gave less non-specific amplification of high molecular weight products. The condition (b) and (c) gave similar results for targets amplified, however, the condition (c) sometimes gave some non-specific bands. Because of the simplicity of condition (c), this procedure can be useful when the starting DNA template is at low concentration.

What is claimed is:

1. An oligonucleotide primer for the detection of a target nucleic acid sequence, said primer comprising 3′ complementary portion and 5′ non-complementary portion, wherein said 5′ non-complementary portion comprises at least one restriction enzyme site, wherein said restriction site acts as detection marker in a process of detecting said target nucleic acid sequence, whereby a detection signal generated from enzymatic manipulation on said restriction site of a reaction product is indicative of the presence of said target nucleic acid sequence.

2. An oligonucleotide primer of claim 1, wherein said restriction site is allele-specific, gene-specific or SNP-specific.

3. A method of analyzing multiple targets in a polynucleotide, said method comprising
(a) providing a set or sets of multiple primers with target nucleic acids in reactions of primer extension or amplification, wherein said reactions produce nucleic acid products in that each nucleic acid fragment comprises at least one restriction site;
(b) digesting said nucleic acid products on said restriction sites with cognate restriction enzymes;
(c) joining digested products of step (b), whereby randomly joined nucleic acid fragments are created;
(d) amplify joined products of step (c); and
(e) detecting amplified products of step (d).

4. The method of claim 3, wherein said primers are oligonucleotides comprising 3′ complementary portion, or 3′ complementary portion and 5′ non-complementary portion.

5. The method of claim 3, wherein said a set or sets of multiple primer comprise mixtures of target specific primers, wherein primer pairs of forward primers and reverse primers specific for each target are included.

6. The method of claim 3, wherein said a set or sets of multiple primer comprise mixtures of target specific reverse primers and universal primer, wherein said universal primers comprise sequence identical or homologous to said non-complementary portion of target specific forward primers.

7. The method of claim 3, wherein said primers or a subset of said primers comprise capture moiety.

8. The method of claim 3, wherein said capture moiety is biotin.

9. The method of claim 3, wherein said primer extension is first strand cDNA synthesis from target RNA in the presence of a set of target specific primers, random primers or oligo dT primers.

10. The method of claim 3, wherein said primer extension is second strand cDNA synthesis in the presence of a set of target specific primers or random primers.

11. The method of claim 3, wherein said amplification is polymerase chain reaction.

12. The method of claim 3, wherein said amplification is carried out at least once for 1 to 30 cycles.

13. The method of claim 3, wherein said amplification is carried out at least once for 3 to 15 cycles.

14. The method of claim 3, further comprising purification and isolation steps before and/or after said step of digesting said nucleic acid products on said restriction sites with cognate restriction enzymes.

15. The method of claim 14, wherein said purification and isolation steps comprise immobilizing said nucleic acid product on a solid support.

16. The method of claim 15, wherein said solid support is streptavidin coated beads.

17. The method of claim 3, wherein said joining is by ligation using a DNA ligase.

18. The method of claim 3, wherein said amplifying is performed using said a set or sets of multiple primers.

19. The method of claim 3, wherein said amplifying is performed using universal primers having sequences identical or homologous to non-complementary portions of target specific primers.

20. The method of claim 19, wherein said universal primers comprise fluorescence dye labels.

21. The method of claim 3, wherein said restriction sites are located on target sequences or on primer sequences, wherein locations of said restriction sites are chosen such that amplification products digested on said restriction site are distinguishable by their sizes and/or labels.

22. The method of claim 3, wherein said restriction sites are the same restriction site for all nucleic acid fragments generated in said reactiion.

23. The method of claim 3, wherein said restriction sites are different and specific for a subset of targets.

24. The method of claim 3, wherein said detecting is electrophoresis.

25. The method of claim 3, wherein said multiple targets comprise SNPs or mutations.

26. The method of claim 3, wherein said a set or sets of multiple primer comprise mixtures of reverse primers and allele-specific forward primers, wherein two allele-specific forward primers and one common reverse primer for each target are included.

27. The method of claim 26, wherein said allele-specific forward primers comprise 3′ends which are complementary to either allele at mutation or polymorphism sites.

28. The method of claim 26, wherein each of said two allele-specific forward primers comprises allele-specific restriction site that is different and specific for each allele and is located 5′ of the complementary portion of said allele-specific forward primers.

29. The method of claim 26, wherein said two allele-specific forward primers comprise the same restriction sites that have different locations.

30. The method of claim 26, wherein said allele-specific forward primers comprise first and second restriction sites in non-complementary portion of each primer, wherein said first restriction sites on all forward primers in a set of multiple primers are the same restriction site and are located 5′ of said second restriction sites, whereas said second restriction sites are allele-specific restriction sites which are different and specific for each of said two allele-specific forward primers and are located 5′ of the complementary portion of said allele-specific forward primers.

31. The method of claim 30, wherein said second restriction sites produce 5′ protruding ends after digestion.

32. The method of claims 3, wherein said step of detecting amplified products comprising
(a) purifying amplified products;
(b) digesting amplified products;
(c) extending with a DNA polymerase in the presence of fluorescence dye labeled terminators; and
(d) putting extended DNA product of step (c) into a electrophoresis system.

33. The method of claim 32, wherein said step of purifying amplified products comprises eliminating dNTP and primers.

34. The method of claim 32, wherein said eliminating dNTP and primers comprises incubating with shrimp alkaline phosphatase and exonuclease I.

35. The method of claim 32, wherein said step of digesting amplified products comprises digesting on said restriction sites.

36. The method of claim 32, wherein said step of digesting amplified products comprises digesting on said first restriction sites.

37. The method of claim 32, wherein said step of digesting amplified products comprises digesting on said second restriction sites.

38. The method of claim 32, wherein said terminators are ddNTP.

39. The method of claim 32, wherein said electrophoresis system is a gel or capillary electrophoresis system.

40. The method of claim 32, wherein said electrophoresis system is DNA sequencer.

41. A method of analyzing multiple targets in a polynucleotide, said method comprising
(a) providing different sets of multiple primers with target nucleic acids in separate reactions of primer extension or amplification, wherein said separate reactions produce nucleic acid products in that each nucleic acid fragment comprises at least one restriction site;
(b) digesting said nucleic acid products of said separate reactions on said restriction sites with cognate restriction enzymes;
(c) joining digested products derived from said separate reactions together, whereby randomly joined nucleic acid fragments from said separated reactions are created;
(d) amplify joined products of step (c); and
(e) detecting amplified products of step (d).

42. A kit for use in the analysis and detection of multiple targets in a polynucleotide, said kit comprising: said a set or sets of multiple primers, said universal primers, said restriction enzymes, said DNA ligase, said DNA polymerase, said ddNTP, buffers for all enzymes, dNTPs.


SEQUENCE LISTING (284 sequences)

SEQ ID NO: 1 - DNA, 25 bp, Artificial
  derived from vector sequence
aaaagtaaaacgacggcgagaattc

SEQ ID NO: 2 - DNA, 21 bp, Artificial
  derived from vector sequence
gcggaaacagctatgaccatg

SEQ ID NO: 3 - DNA, 52 bp, Homo sapiens
gtaaaacgacggcgagaattctctccggagttcatattctatgaggtatcac

SEQ ID NO: 4 - DNA, 53 bp, Homo sapiens
gtaaaacgacggcgagaattcgacgatcgagttcatattctatgaggtatcat

SEQ ID NO: 5 - DNA, 43 bp, Homo sapiens
gcggaaacagctatgaccatggaatgaggacagccatagagac

SEQ ID NO: 6 - DNA, 22 bp, Homo sapiens
ctgtaggtgtggcttgttggga

SEQ ID NO: 7 - DNA, 48 bp, Homo sapiens
gtaaaacgacggcgagaattctctccggctgaaaggaagactcagagg

SEQ ID NO: 8 - DNA, 49 bp, Homo sapiens
gtaaaacgacggcgagaattcgacgatcgctgaaaggaagactcagagc

SEQ ID NO: 9 - DNA, 40 bp, Homo sapiens
gcggaaacagctatgaccatgcctggacagttactcacag

SEQ ID NO: 10 - DNA, 20 bp, Homo sapiens
cctgattagcaccccaagtc

SEQ ID NO: 11 - DNA, 46 bp, Homo sapiens
gtaaaacgacggcgagaattctctccggacttctggtttgctcttt

SEQ ID NO: 12 - DNA, 48 bp, Homo sapiens
gtaaaacgacggcgagaattcgacgatcggacttctggtttgctcttc

SEQ ID NO: 13 - DNA, 43 bp, Homo sapiens
gcggaaacagctatgaccatgcagagctcaggaggagttaatg

SEQ ID NO: 14 - DNA, 26 bp, Homo sapiens
ataaatgtcactgttagagccatcaa

SEQ ID NO: 15 - DNA, 50 bp, Homo sapiens
gtaaaacgacggcgagaattctctccggcattggaagaacacccaagcaa

SEQ ID NO: 16 - DNA, 49 bp, Homo sapiens
gtaaaacgacggcgagaattcgacgatcattggaagaacacccaagcac

SEQ ID NO: 17 - DNA, 41 bp, Homo sapiens
gcggaaacagctatgaccatggacagcctgtccactcatgc

SEQ ID NO: 18 - DNA, 20 bp, Homo sapiens
cttgcatcactgagtccctg

SEQ ID NO: 19 - DNA, 49 bp, Homo sapiens
gtaaaacgacggcgagaattctctccggctgaggcaaacttgaggttct

SEQ ID NO: 20 - DNA, 48 bp, Homo sapiens
gtaaaacgacggcgagaattcgacgatctgaggcaaacttgaggttcc

SEQ ID NO: 21 - DNA, 43 bp, Homo sapiens
gcggaaacagctatgaccatgggaaatgctttgtccttccgta

SEQ ID NO: 22 - DNA, 20 bp, Homo sapiens
agggcccacacctctgcctt

SEQ ID NO: 23 - DNA, 54 bp, Homo sapiens
gtaaaacgacggcgagaattctctccggtgttatttctatctgatttcttgaac

SEQ ID NO: 24 - DNA, 55 bp, Homo sapiens
gtaaaacgacggcgagaattcgacgatcgtgttatttctatctgatttcttgaag

SEQ ID NO: 25 - DNA, 42 bp, Homo sapiens
gcggaaacagctatgaccatggctagggtctcttaggataac

SEQ ID NO: 26 - DNA, 23 bp, Homo sapiens
caatgccctaatctctttgcctt

SEQ ID NO: 27 - DNA, 51 bp, Homo sapiens
gtaaaacgacggcgagaattctctccggccaggtgtcactgaagattgtat

SEQ ID NO: 28 - DNA, 50 bp, Homo sapiens
gtaaaacgacggcgagaattcgacgatccaggtgtcactgaagattgtac

SEQ ID NO: 29 - DNA, 42 bp, Homo sapiens
gcggaaacagctatgaccatgcttttccctggtcctaactgc

SEQ ID NO: 30 - DNA, 21 bp, Homo sapiens
gtgttgcctgagtcaccagtg

SEQ ID NO: 31 - DNA, 53 bp, Homo sapiens
gtaaaacgacggcgagaattctctccggcataatcacaatgctattattatgc

SEQ ID NO: 32 - DNA, 46 bp, Homo sapiens
gtaaaacgacggcgagaattccatgatcacaatgctattattatgt

SEQ ID NO: 33 - DNA, 44 bp, Homo sapiens
gcggaaacagctatgaccatgattctttgaagattagacgcatg

SEQ ID NO: 34 - DNA, 27 bp, Homo sapiens
ctttgtctatctgttgcactaacctat

SEQ ID NO: 35 - DNA, 47 bp, Homo sapiens
gtaaaacgacggcgagaattctctccggtaacatacaactgaagcca

SEQ ID NO: 36 - DNA, 48 bp, Homo sapiens
gtaaaacgacggcgagaattcgacgatcgtaacatacaactgaagccc

SEQ ID NO: 37 - DNA, 43 bp, Homo sapiens
gcggaaacagctatgaccatgcagtttgtcctcatcctacttg

SEQ ID NO: 38 - DNA, 22 bp, Homo sapiens
caggcaggctgtttccacatga

SEQ ID NO: 39 - DNA, 49 bp, Homo sapiens
gtaaaacgacggcgagaattctctccggactagcagattcacggttgaa

SEQ ID NO: 40 - DNA, 50 bp, Homo sapiens
gtaaaacgacggcgagaattcgacgatcgactagcagattcacggttgag

SEQ ID NO: 41 - DNA, 41 bp, Homo sapiens
gcggaaacagctatgaccatggaagaagaaaggctgatggc

SEQ ID NO: 42 - DNA, 24 bp, Homo sapiens
gcaactcatctttgatgggtcatg

SEQ ID NO: 43 - DNA, 42 bp, Homo sapiens
gtaaaacgacggcgagaattcccctatcccccagtttgtttc

SEQ ID NO: 44 - DNA, 43 bp, Homo sapiens
gcggaaacagctatgaccatggttgcgtgactgtgaggtttgc

SEQ ID NO: 45 - DNA, 22 bp, Homo sapiens
tgttggagtgtttgggctaagt

SEQ ID NO: 46 - DNA, 43 bp, Homo sapiens
gtaaaacgacggcgagaattccagtcccagaaacactcactgg

SEQ ID NO: 47 - DNA, 43 bp, Homo sapiens
gcggaaacagctatgaccatgcatgggcctaagtctgcatttc

SEQ ID NO: 48 - DNA, 21 bp, Homo sapiens
gatgggatctttggctgagag

SEQ ID NO: 49 - DNA, 43 bp, Homo sapiens
gtaaaacgacggcgagaattcaatgaggccattttgccataca

SEQ ID NO: 50 - DNA, 41 bp, Homo sapiens
gcggaaacagctatgaccatgcaattggcttgctctatgct

SEQ ID NO: 51 - DNA, 25 bp, Homo sapiens
ctcccaatgaaccttaaatgcacga

SEQ ID NO: 52 - DNA, 44 bp, Homo sapiens
gtaaaacgacggcgagaattcgcatcacagaggagatgtatcag

SEQ ID NO: 53 - DNA, 42 bp, Homo sapiens
gcggaaacagctatgaccatggtgagcccaacagagctgtct

SEQ ID NO: 54 - DNA, 24 bp, Homo sapiens
ttgcattagaaaggtgggcatttg

SEQ ID NO: 55 - DNA, 42 bp, Homo sapiens
gtaaaacgacggcgagaattcctcctcctcacttacctgaat

SEQ ID NO: 56 - DNA, 42 bp, Homo sapiens
gcggaaacagctatgaccatggctgaatttccagcctgtcta

SEQ ID NO: 57 - DNA, 23 bp, Homo sapiens
gcggctagtttctaattccctgt

SEQ ID NO: 58 - DNA, 41 bp, Homo sapiens
gtaaaacgacggcgagaattcgctggaaattcagccacctg

SEQ ID NO: 59 - DNA, 44 bp, Homo sapiens
gcggaaacagctatgaccatgttttgctaacaaaacgggagaca

SEQ ID NO: 60 - DNA, 23 bp, Homo sapiens
atgcccacctttctaatgcaaat

SEQ ID NO: 61 - DNA, 42 bp, Homo sapiens
gtaaaacgacggcgagaattcaccctactccaaggtggctgt

SEQ ID NO: 62 - DNA, 39 bp, Homo sapiens
gcggaaacagctatgaccatgagacccagccttggctgt

SEQ ID NO: 63 - DNA, 20 bp, Homo sapiens
cagaagcctggagcctttcc

SEQ ID NO: 64 - DNA, 43 bp, Homo sapiens
gtaaaacgacggcgagaattcttgttaggggaggccaacatgc

SEQ ID NO: 65 - DNA, 41 bp, Homo sapiens
gcggaaacagctatgaccatggcttaggaaaggcactggtc

SEQ ID NO: 66 - DNA, 22 bp, Homo sapiens
gtgtatgcttacgcacgcactg

SEQ ID NO: 67 - DNA, 43 bp, Homo sapiens
gtaaaacgacggcgagaattctgctccttaagggatgttccaa

SEQ ID NO: 68 - DNA, 42 bp, Homo sapiens
gcggaaacagctatgaccatgtacaacacccatgcaggcatg

SEQ ID NO: 69 - DNA, 23 bp, Homo sapiens
cccctgcaatacatgacacctgt

SEQ ID NO: 70 - DNA, 45 bp, Homo sapiens
gtaaaacgacggcgagaattcgggcatgttctgattcctcattac

SEQ ID NO: 71 - DNA, 44 bp, Homo sapiens
gcggaaacagctatgaccatgcaccactcaatgaatttcccatg

SEQ ID NO: 72 - DNA, 20 bp, Homo sapiens
gcaggcatgcatacagccta

SEQ ID NO: 73 - DNA, 42 bp, Homo sapiens
gtaaaacgacggcgagaattcccatgtacgcttcgctcagtt

SEQ ID NO: 74 - DNA, 43 bp, Homo sapiens
gcggaaacagctatgaccatgttctccggaggcaggattccga

SEQ ID NO: 75 - DNA, 23 bp, Homo sapiens
cttcattgttggcgtctccactt

SEQ ID NO: 76 - DNA, 44 bp, Homo sapiens
gtaaaacgacggcgagaattcaatgtaatgagatgggcctggtg

SEQ ID NO: 77 - DNA, 42 bp, Homo sapiens
gcggaaacagctatgaccatgacatcttgggtcttccgcatt

SEQ ID NO: 78 - DNA, 22 bp, Homo sapiens
gagcaccctgacgcagtcttag

SEQ ID NO: 79 - DNA, 43 bp, Homo sapiens
gtaaaacgacggcgagaattcacgcatgccaattaagggttcg

SEQ ID NO: 80 - DNA, 42 bp, Homo sapiens
gcggaaacagctatgaccatgagtgcgtggtcgctaagcttc

SEQ ID NO: 81 - DNA, 21 bp, Homo sapiens
gagcaacaggactggtggttg

SEQ ID NO: 82 - DNA, 43 bp, Homo sapiens
gtaaaacgacggcgagaattcaaagacggtactgcctgcttcc

SEQ ID NO: 83 - DNA, 41 bp, Homo sapiens
gcggaaacagctatgaccatgagcaacaggactggtggttg

SEQ ID NO: 84 - DNA, 22 bp, Homo sapiens
gcactgcacagatccccagata

SEQ ID NO: 85 - DNA, 41 bp, Homo sapiens
gtaaaacgacggcgagaattcgcagtgagctatgaccacac

SEQ ID NO: 86 - DNA, 42 bp, Homo sapiens
gcggaaacagctatgaccatggtggagaccaaggtcaaaatc

SEQ ID NO: 87 - DNA, 23 bp, Homo sapiens
cagagcttccttggggtgactgg

SEQ ID NO: 88 - DNA, 42 bp, Homo sapiens
gtaaaacgacggcgagaattcaggcctgaactcctccttgtg

SEQ ID NO: 89 - DNA, 45 bp, Homo sapiens
gcggaaacagctatgaccatgattcagctctggttggaactgctc

SEQ ID NO: 90 - DNA, 21 bp, Homo sapiens
gacccgagactgaagccagat

SEQ ID NO: 91 - DNA, 41 bp, Homo sapiens
gtaaaacgacggcgagaattcacatgtcctcagtggcttcc

SEQ ID NO: 92 - DNA, 41 bp, Homo sapiens
gcggaaacagctatgaccatgctccttcgatactcccgatg

SEQ ID NO: 93 - DNA, 21 bp, Homo sapiens
gtgagaggacgaggcaccttt

SEQ ID NO: 94 - DNA, 41 bp, Homo sapiens
gtaaaacgacggcgagaattcgagggggagccaacatccgt

SEQ ID NO: 95 - DNA, 41 bp, Homo sapiens
gcggaaacagctatgaccatgtgactttgagaaaccacgct

SEQ ID NO: 96 - DNA, 21 bp, Homo sapiens
agcgttgccaacaggcatcac

SEQ ID NO: 97 - DNA, 41 bp, Homo sapiens
gtaaaacgacggcgagaattcctcctgcctctttcaggtgt

SEQ ID NO: 98 - DNA, 45 bp, Homo sapiens
gcggaaacagctatgaccatggaaaaattgctggggttggtcaag

SEQ ID NO: 99 - DNA, 21 bp, Homo sapiens
ccattttacggccttcctcag

SEQ ID NO: 100 - DNA, 41 bp, Homo sapiens
gtaaaacgacggcgagaattcagtatgccaagttgctgcat

SEQ ID NO: 101 - DNA, 41 bp, Homo sapiens
gcggaaacagctatgaccatggcaaggcctgtttggatgag

SEQ ID NO: 102 - DNA, 22 bp, Homo sapiens
ggccatatcgtcacactttctg

SEQ ID NO: 103 - DNA, 45 bp, Homo sapiens
gtaaaacgacggcgagaattctacatgcatagtcctagccagtta

SEQ ID NO: 104 - DNA, 45 bp, Homo sapiens
gcggaaacagctatgaccatgggcagaatgaatcaatttaacttc

SEQ ID NO: 105 - DNA, 27 bp, Homo sapiens
tttcaacaagtaccctagacctcactt

SEQ ID NO: 106 - DNA, 48 bp, Homo sapiens
gtaaaacgacggcgagaattctttaactggctaggactatgcatgtag

SEQ ID NO: 107 - DNA, 42 bp, Homo sapiens
gcggaaacagctatgaccatgtctacccagtctacctcagga

SEQ ID NO: 108 - DNA, 25 bp, Homo sapiens
ggttaatggcctcagggtaaccttt

SEQ ID NO: 109 - DNA, 42 bp, Homo sapiens
gtaaaacgacggcgagaattctcttacaaacggctgcaacac

SEQ ID NO: 110 - DNA, 45 bp, Homo sapiens
gcggaaacagctatgaccatgtgcttatgtgttctgccttgatac

SEQ ID NO: 111 - DNA, 24 bp, Homo sapiens
cactctagcaagtccaagtgtggt

SEQ ID NO: 112 - DNA, 44 bp, Homo sapiens
gtaaaacgacggcgagaattcaagcttggcatatctgggagaca

SEQ ID NO: 113 - DNA, 43 bp, Homo sapiens
gcggaaacagctatgaccatgtacttctttccatctgcactgt

SEQ ID NO: 114 - DNA, 24 bp, Homo sapiens
tggaaccagccttcattttcatac

SEQ ID NO: 115 - DNA, 42 bp, Homo sapiens
gtaaaacgacggcgagaattccataagcacagcccaaatcag

SEQ ID NO: 116 - DNA, 44 bp, Homo sapiens
gcggaaacagctatgaccatgatgttgaacctcttttcctgtgc

SEQ ID NO: 117 - DNA, 23 bp, Homo sapiens
tgcctaatgggtaatggctaatg

SEQ ID NO: 118 - DNA, 39 bp, Homo sapiens
gtaaaacgacggcgagaattcctatagcttacctctccc

SEQ ID NO: 119 - DNA, 42 bp, Homo sapiens
gcggaaacagctatgaccatgctgcttgcgtggaggcttttc

SEQ ID NO: 120 - DNA, 22 bp, Homo sapiens
gcagtgctctggagaaatgatg

SEQ ID NO: 121 - DNA, 43 bp, Homo sapiens
gtaaaacgacggcgagaattcacctcgattggcacatcaatct

SEQ ID NO: 122 - DNA, 44 bp, Homo sapiens
gcggaaacagctatgaccatgcggtgtttacttgccaagtgcta

SEQ ID NO: 123 - DNA, 25 bp, Homo sapiens
gcattggtccagtgaacgctatcaa

SEQ ID NO: 124 - DNA, 43 bp, Homo sapiens
gtaaaacgacggcgagaattctttcctggacttggtggatttc

SEQ ID NO: 125 - DNA, 43 bp, Homo sapiens
gcggaaacagctatgaccatggcactcagcatcaattgtcaac

SEQ ID NO: 126 - DNA, 21 bp, Homo sapiens
ggcaggagtgttcatccattg

SEQ ID NO: 127 - DNA, 42 bp, Homo sapiens
gtaaaacgacggcgagaattctggagtcccaacaaatcgaga

SEQ ID NO: 128 - DNA, 43 bp, Homo sapiens
gcggaaacagctatgaccatggatcttgtgtcgcccacttcaa

SEQ ID NO: 129 - DNA, 21 bp, Homo sapiens
ctttgccagtgcatcttaccg

SEQ ID NO: 130 - DNA, 45 bp, Homo sapiens
gtaaaacgacggcgagaattcaattggaatttgaacccaatcctg

SEQ ID NO: 131 - DNA, 43 bp, Homo sapiens
gcggaaacagctatgaccatgaaatcgccacatttcacacagg

SEQ ID NO: 132 - DNA, 21 bp, Homo sapiens
cggtaagatgcactggcaaag

SEQ ID NO: 133 - DNA, 42 bp, Homo sapiens
gtaaaacgacggcgagaattcagtgggagagaaggccaactg

SEQ ID NO: 134 - DNA, 40 bp, Homo sapiens
gcggaaacagctatgaccatggtggagacagaagggttga

SEQ ID NO: 135 - DNA, 23 bp, Homo sapiens
ctgaagtcttccattggcgaaag

SEQ ID NO: 136 - DNA, 42 bp, Homo sapiens
gtaaaacgacggcgagaattcggcagagaatgtgctggtgac

SEQ ID NO: 137 - DNA, 44 bp, Homo sapiens
gcggaaacagctatgaccatgtgctttagagagataagccttcc

SEQ ID NO: 138 - DNA, 22 bp, Homo sapiens
caaccagggagtccaccaagtc

SEQ ID NO: 139 - DNA, 43 bp, Homo sapiens
gtaaaacgacggcgagaattcgacggtgaagactacaggcaca

SEQ ID NO: 140 - DNA, 45 bp, Homo sapiens
gcggaaacagctatgaccatgctccaaagaggtaaagtcagagct

SEQ ID NO: 141 - DNA, 24 bp, Homo sapiens
aatcttaaagaccaagcccagttg

SEQ ID NO: 142 - DNA, 43 bp, Homo sapiens
gtaaaacgacggcgagaattctgtgcctgtagtcttcaccgtc

SEQ ID NO: 143 - DNA, 42 bp, Homo sapiens
gcggaaacagctatgaccatgcccaccatatgtttgcttcgt

SEQ ID NO: 144 - DNA, 24 bp, Homo sapiens
ctaggggacatagagaacgcaaat

SEQ ID NO: 145 - DNA, 42 bp, Homo sapiens
gtaaaacgacggcgagaattcggggcatttaggccaggatag

SEQ ID NO: 146 - DNA, 42 bp, Homo sapiens
gcggaaacagctatgaccatggagggcctctctaaagggcat

SEQ ID NO: 147 - DNA, 21 bp, Homo sapiens
actccaccctggaggaaaaca

SEQ ID NO: 148 - DNA, 43 bp, Homo sapiens
gtaaaacgacggcgagaattctgagcttccagtgatggttgaa

SEQ ID NO: 149 - DNA, 46 bp, Homo sapiens
gcggaaacagctatgaccatgcctgtactttagatatttgtggcta

SEQ ID NO: 150 - DNA, 22 bp, Homo sapiens
aagatagcaggattgccggaag

SEQ ID NO: 151 - DNA, 46 bp, Homo sapiens
gtaaaacgacggcgagaattcatcatttctatgtcagaagggcaaa

SEQ ID NO: 152 - DNA, 46 bp, Homo sapiens
gcggaaacagctatgaccatgtgctagatatgctcctttgagtaag

SEQ ID NO: 153 - DNA, 23 bp, Homo sapiens
cagaggtgtcagtgaagacctga

SEQ ID NO: 154 - DNA, 42 bp, Homo sapiens
gtaaaacgacggcgagaattctcctgaagcctgaaggtagcc

SEQ ID NO: 155 - DNA, 44 bp, Homo sapiens
gcggaaacagctatgaccatgtcccaggattaaagcacatgcaa

SEQ ID NO: 156 - DNA, 24 bp, Homo sapiens
gctaccacagatgcctagcccaat

SEQ ID NO: 157 - DNA, 43 bp, Homo sapiens
gtaaaacgacggcgagaattctgccctcggtggatattcagat

SEQ ID NO: 158 - DNA, 47 bp, Homo sapiens
gcggaaacagctatgaccatgaaactttgccgttctgatttcatcta

SEQ ID NO: 159 - DNA, 22 bp, Homo sapiens
caagcaccacagtgtgccaagt

SEQ ID NO: 160 - DNA, 43 bp, Homo sapiens
gtaaaacgacggcgagaattccctgagccttttggctctacta

SEQ ID NO: 161 - DNA, 45 bp, Homo sapiens
gcggaaacagctatgaccatgattgtataccacaatggtatggct

SEQ ID NO: 162 - DNA, 22 bp, Homo sapiens
ggtgctgcatgtgagtggaaca

SEQ ID NO: 163 - DNA, 46 bp, Homo sapiens
gtaaaacgacggcgagaattcccatttgtcattgagaaaaaaaacc

SEQ ID NO: 164 - DNA, 47 bp, Homo sapiens
gcggaaacagctatgaccatgacttctaggcacataaaaaaccgtgg

SEQ ID NO: 165 - DNA, 24 bp, Homo sapiens
gattgggattctgccgaaagactg

SEQ ID NO: 166 - DNA, 41 bp, Homo sapiens
gtaaaacgacggcgagaattcccaggtgccaggtcttctaa

SEQ ID NO: 167 - DNA, 47 bp, Homo sapiens
gcggaaacagctatgaccatgctaaactcatacatgctgttttcatc

SEQ ID NO: 168 - DNA, 25 bp, Homo sapiens
gaatcgttatggcctggctaaactc

SEQ ID NO: 169 - DNA, 43 bp, Homo sapiens
gtaaaacgacggcgagaattctccatttccaaacccaaatcct

SEQ ID NO: 170 - DNA, 42 bp, Homo sapiens
gcggaaacagctatgaccatgttcactccgggtcaagttgtg

SEQ ID NO: 171 - DNA, 23 bp, Homo sapiens
gcccagccctcctttggttagtg

SEQ ID NO: 172 - DNA, 43 bp, Homo sapiens
gtaaaacgacggcgagaattctcagtagtctggtgggactgtt

SEQ ID NO: 173 - DNA, 44 bp, Homo sapiens
gcggaaacagctatgaccatgccacaccgaaattcttgaggtag

SEQ ID NO: 174 - DNA, 22 bp, Homo sapiens
gcatgacagcagtgcatggaag

SEQ ID NO: 175 - DNA, 44 bp, Homo sapiens
gtaaaacgacggcgagaattctgtgttttgcatggcggtactct

SEQ ID NO: 176 - DNA, 46 bp, Homo sapiens
gcggaaacagctatgaccatggaggattctaaagtctgaattgaca

SEQ ID NO: 177 - DNA, 22 bp, Homo sapiens
gcatgacagcagtgcatggaag

SEQ ID NO: 178 - DNA, 44 bp, Homo sapiens
gtaaaacgacggcgagaattcgggaggagaggacctggctgaaa

SEQ ID NO: 179 - DNA, 42 bp, Homo sapiens
gcggaaacagctatgaccatgggaactagcgagaacgaggaa

SEQ ID NO: 180 - DNA, 24 bp, Homo sapiens
cgacagcctgcctatttccaaagg

SEQ ID NO: 181 - DNA, 41 bp, Homo sapiens
gtaaaacgacggcgagaattctctccggcattagggatttg

SEQ ID NO: 182 - DNA, 44 bp, Homo sapiens
gcggaaacagctatgaccatgggagacagacgtttaaccggtga

SEQ ID NO: 183 - DNA, 22 bp, Homo sapiens
cccagctgtgagtgttgtgtgg

SEQ ID NO: 184 - DNA, 43 bp, Homo sapiens
gtaaaacgacggcgagaattccattcctgctgtgcccaagagt

SEQ ID NO: 185 - DNA, 43 bp, Homo sapiens
gcggaaacagctatgaccatgccagcttcgaggacatccctct

SEQ ID NO: 186 - DNA, 22 bp, Homo sapiens
ccgtgattccctggaaaggaag

SEQ ID NO: 187 - DNA, 42 bp, Homo sapiens
gtaaaacgacggcgagaattcgtcttcgaccccatcttcgtc

SEQ ID NO: 188 - DNA, 42 bp, Homo sapiens
gcggaaacagctatgaccatgctgacagccgaatgacctctg

SEQ ID NO: 189 - DNA, 21 bp, Homo sapiens
gccggggactgaaaactctta

SEQ ID NO: 190 - DNA, 43 bp, Homo sapiens
gtaaaacgacggcgagaattcaccttttctacgcgacctttgg

SEQ ID NO: 191 - DNA, 43 bp, Homo sapiens
gcggaaacagctatgaccatggctcctagctcctttcaggaag

SEQ ID NO: 192 - DNA, 21 bp, Homo sapiens
tatgcctgtcggctcacagat

SEQ ID NO: 193 - DNA, 42 bp, Homo sapiens
gtaaaacgacggcgagaattcaacatggcaggtgctgttagc

SEQ ID NO: 194 - DNA, 43 bp, Homo sapiens
gcggaaacagctatgaccatgagaggcaaagctgcagttgtgt

SEQ ID NO: 195 - DNA, 21 bp, Homo sapiens
gcctgtgggtaactggtcaca

SEQ ID NO: 196 - DNA, 43 bp, Homo sapiens
gtaaaacgacggcgagaattcaagggaggcctggttcacctac

SEQ ID NO: 197 - DNA, 43 bp, Homo sapiens
gcggaaacagctatgaccatggctctcccagagatgcgctcat

SEQ ID NO: 198 - DNA, 22 bp, Homo sapiens
gaagcagaaactcggcttgagg

SEQ ID NO: 199 - DNA, 43 bp, Homo sapiens
gtaaaacgacggcgagaattcgagggcaaagttgccactacct

SEQ ID NO: 200 - DNA, 44 bp, Homo sapiens
gcggaaacagctatgaccatgacatttgatcgctccaccaaggt

SEQ ID NO: 201 - DNA, 22 bp, Homo sapiens
attaggggtctctgggctgaag

SEQ ID NO: 202 - DNA, 45 bp, Homo sapiens
gtaaaacgacggcgagaattctgatggcccctccatcgattagta

SEQ ID NO: 203 - DNA, 43 bp, Homo sapiens
gcggaaacagctatgaccatggctgacatcaacggccaatacc

SEQ ID NO: 204 - DNA, 24 bp, Homo sapiens
gcaggccgtttgccttcaagatag

SEQ ID NO: 205 - DNA, 44 bp, Homo sapiens
gtaaaacgacggcgagaattcattgtgccaagctgctaatggtc

SEQ ID NO: 206 - DNA, 43 bp, Homo sapiens
gcggaaacagctatgaccatggagttcagatgcaccacggagt

SEQ ID NO: 207 - DNA, 22 bp, Homo sapiens
gaggctggatgtgccaagtacc

SEQ ID NO: 208 - DNA, 44 bp, Homo sapiens
gtaaaacgacggcgagaattcctttggttgcatgtcgatgtaag

SEQ ID NO: 209 - DNA, 45 bp, Homo sapiens
gcggaaacagctatgaccatgtaccagttgtgaatggctggctag

SEQ ID NO: 210 - DNA, 21 bp, Homo sapiens
cccagaggatgttccaggtct

SEQ ID NO: 211 - DNA, 45 bp, Homo sapiens
gtaaaacgacggcgagaattctgattggatgcctcacctacttgc

SEQ ID NO: 212 - DNA, 43 bp, Homo sapiens
gcggaaacagctatgaccatgtcacctggaaccctcacaagat

SEQ ID NO: 213 - DNA, 22 bp, Homo sapiens
ccgagactgcctgcaaaacatt

SEQ ID NO: 214 - DNA, 40 bp, Homo sapiens
gtaaaacgacggcgagaattcaggagcttccctcagcagt

SEQ ID NO: 215 - DNA, 43 bp, Homo sapiens
gcggaaacagctatgaccatgaaacagggcacagtgggatttg

SEQ ID NO: 216 - DNA, 22 bp, Homo sapiens
ttcccatagacagggacagcat

SEQ ID NO: 217 - DNA, 41 bp, Homo sapiens
gtaaaacgacggcgagaattcgctgacagtgaggtcgattc

SEQ ID NO: 218 - DNA, 42 bp, Homo sapiens
gcggaaacagctatgaccatgcttgtccaacaactccaggaa

SEQ ID NO: 219 - DNA, 23 bp, Homo sapiens
ggagctaaacatgctgccaacca

SEQ ID NO: 220 - DNA, 42 bp, Homo sapiens
gtaaaacgacggcgagaattcgccaatggctccacttgagtt

SEQ ID NO: 221 - DNA, 41 bp, Homo sapiens
gcggaaacagctatgaccatggaagaggcttctccaccttg

SEQ ID NO: 222 - DNA, 22 bp, Homo sapiens
gggctatgtgcatggagctttc

SEQ ID NO: 223 - DNA, 45 bp, Homo sapiens
gtaaaacgacggcgagaattcagatagaggagcaccaggctgaca

SEQ ID NO: 224 - DNA, 42 bp, Homo sapiens
gcggaaacagctatgaccatgcacatagcccagcaaagagca

SEQ ID NO: 225 - DNA, 21 bp, Homo sapiens
gccaacttccaaggtggagaa

SEQ ID NO: 226 - DNA, 42 bp, Homo sapiens
gtaaaacgacggcgagaattcgcagatccccctgaaattacg

SEQ ID NO: 227 - DNA, 44 bp, Homo sapiens
gcggaaacagctatgaccatgcacattggatgaagcccgtcttc

SEQ ID NO: 228 - DNA, 21 bp, Homo sapiens
gccaaatctacttccccagca

SEQ ID NO: 229 - DNA, 44 bp, Homo sapiens
gtaaaacgacggcgagaattcgtgttccttgcatcactgagtcc

SEQ ID NO: 230 - DNA, 46 bp, Homo sapiens
gcggaaacagctatgaccatgaaggcatgttctgccacttaatttc

SEQ ID NO: 231 - DNA, 21 bp, Homo sapiens
gcagccagcactctgtcctaa

SEQ ID NO: 232 - DNA, 43 bp, Homo sapiens
gtaaaacgacggcgagaattctgggaaatgctttgtccttccg

SEQ ID NO: 233 - DNA, 41 bp, Homo sapiens
gcggaaacagctatgaccatggctgtaccttcctgggaacc

SEQ ID NO: 234 - DNA, 23 bp, Homo sapiens
gacctgggttaaagccatggaaa

SEQ ID NO: 235 - DNA, 43 bp, Homo sapiens
gtaaaacgacggcgagaattcaaaatatggctgtggaagatga

SEQ ID NO: 236 - DNA, 42 bp, Homo sapiens
gcggaaacagctatgaccatgaggtgccttgatgtggtagat

SEQ ID NO: 237 - DNA, 23 bp, Homo sapiens
ggcaggtggctttgttggactgt

SEQ ID NO: 238 - DNA, 42 bp, Homo sapiens
gtaaaacgacggcgagaattccctctgggcacttgtttgcta

SEQ ID NO: 239 - DNA, 45 bp, Homo sapiens
gcggaaacagctatgaccatgtattgggctagggtctcttaggat

SEQ ID NO: 240 - DNA, 23 bp, Homo sapiens
gcagtgagatcagcagccccttc

SEQ ID NO: 241 - DNA, 42 bp, Homo sapiens
gtaaaacgacggcgagaattccctctgacagcctcgagtgaa

SEQ ID NO: 242 - DNA, 43 bp, Homo sapiens
gcggaaacagctatgaccatgcagctttggtgggagcactttc

SEQ ID NO: 243 - DNA, 23 bp, Homo sapiens
ctgatgggtgttgcctgagtcac

SEQ ID NO: 244 - DNA, 44 bp, Homo sapiens
gtaaaacgacggcgagaattccagcctggttcaagcatattctg

SEQ ID NO: 245 - DNA, 43 bp, Homo sapiens
gcggaaacagctatgaccatgatgaaagcagtgtcatcactgc

SEQ ID NO: 246 - DNA, 22 bp, Homo sapiens
cctgcctcaagtcacccttatg

SEQ ID NO: 247 - DNA, 44 bp, Homo sapiens
gtaaaacgacggcgagaattcggtgcccagcccaatttgattat

SEQ ID NO: 248 - DNA, 44 bp, Homo sapiens
gcggaaacagctatgaccatgcaggggagatgggctgaaaacat

SEQ ID NO: 249 - DNA, 21 bp, Homo sapiens
gccacccacctaatggctcta

SEQ ID NO: 250 - DNA, 43 bp, Homo sapiens
gtaaaacgacggcgagaattcaggcctgcttgcaactcatctt

SEQ ID NO: 251 - DNA, 44 bp, Homo sapiens
gcggaaacagctatgaccatgcctttcagctgaaagcacatatg

SEQ ID NO: 252 - DNA, 23 bp, Homo sapiens
ggatctccaaacacgaccctcca

SEQ ID NO: 253 - DNA, 24 bp, Artificial
  derived from vector sequence
aaaagtggaacgacggcgagaatt

SEQ ID NO: 254 - DNA, 21 bp, Artificial
  derived from vector sequence
gcggaaacagctatgaccatg

SEQ ID NO: 255 - DNA, 42 bp, Vibrio cholerae
gtggaacgacggcgagaattcggggcatacagtcctcatcca

SEQ ID NO: 256 - DNA, 42 bp, Vibrio cholerae
gcggaaacagctatgaccatgggaaacctgccaatccataac

SEQ ID NO: 257 - DNA, 41 bp, Vibrio cholerae
gtggaacgacggcgagactcttccctccaagctctatgctc

SEQ ID NO: 258 - DNA, 43 bp, Vibrio cholerae
gtggaacgacggcgagaattcgacgaagatcattgacgacctc

SEQ ID NO: 259 - DNA, 42 bp, Vibrio cholerae
gcggaaacagctatgaccatgccgcttcatcgtcgttatgtg

SEQ ID NO: 260 - DNA, 41 bp, Vibrio cholerae
gtggaacgacggcgagagataggtggtgtgatgctgctcag

SEQ ID NO: 261 - DNA, 45 bp, Salmonella typhi
gtggaacgacggcgagaattcccttgcacgtttttggttgacaga

SEQ ID NO: 262 - DNA, 42 bp, Salmonella typhi
gcggaaacagctatgaccatggatagcgcggctcacgtactc

SEQ ID NO: 263 - DNA, 41 bp, Salmonella typhi
gtggaacgacggcgagactgaatccggcaataacagatagc

SEQ ID NO: 264 - DNA, 43 bp, Salmonella species
gtggaacgacggcgagaattcggtgaaattatcgccacgttcg

SEQ ID NO: 265 - DNA, 43 bp, Salmonella species
gcggaaacagctatgaccatgcaccgtcaaaggaaccgtaaag

SEQ ID NO: 266 - DNA, 37 bp, Salmonella species
gtggaacgacggcgagggtcatccccaccgaaatacc

SEQ ID NO: 267 - DNA, 44 bp, Shigella dysenteriae
gtggaacgacggcgagaattcgactgataccatggtgcaaaagc

SEQ ID NO: 268 - DNA, 44 bp, Shigella dysenteriae
gcggaaacagctatgaccatgctcgggattggcagcctttaatc

SEQ ID NO: 269 - DNA, 44 bp, Shigella dysenteriae
gtggaacgacggcgagagggtaagttctcttcagaccctgaagg

SEQ ID NO: 270 - DNA, 49 bp, Shigella species
gtggaacgacggcgagaattcacggcttctgaccatagcttcggcagtg

SEQ ID NO: 271 - DNA, 43 bp, Shigella species
gcggaaacagctatgaccatgcgataatgataccggcgctctg

SEQ ID NO: 272 - DNA, 41 bp, Shigella species
gtggaacgacggcgagatcctggtccatcaggcatcagaag

SEQ ID NO: 273 - DNA, 42 bp, E. coli O157
gtggaacgacggcgagaattcggcttcaagatctttggcaac

SEQ ID NO: 274 - DNA, 43 bp, E. coli O157
gcggaaacagctatgaccatgcagtttgtcgaaatggcagcag

SEQ ID NO: 275 - DNA, 41 bp, E. coli O157
gtggaacgacggcgagagcaacaggagtccaatactcagtc

SEQ ID NO: 276 - DNA, 43 bp, E. coli O157
gtggaacgacggcgagaattcagccgttacgatctggttcagc

SEQ ID NO: 277 - DNA, 43 bp, E. coli O157
gcggaaacagctatgaccatgcctggtagtcttgtgcgctttg

SEQ ID NO: 278 - DNA, 39 bp, E. coli O157
gtggaacgacggcgagactgccaccttgcacataagcag

SEQ ID NO: 279 - DNA, 45 bp, Listeria monocytogenes
gtggaacgacggcgagaattctaccaattgcgcaacaaactgaag

SEQ ID NO: 280 - DNA, 45 bp, Listeria monocytogenes
gcggaaacagctatgaccatggctcgaaattgcattcacaacttg

SEQ ID NO: 281 - DNA, 42 bp, Listeria monocytogenes
gtggaacgacggcgagattagtcattcctggcaaatcaatgc

SEQ ID NO: 282 - DNA, 46 bp, Listeria monocytogenes
gtggaacgacggcgagaattcgtgatgatggcgattatgaaaaacc

SEQ ID NO: 283 - DNA, 46 bp, Listeria monocytogenes
gcggaaacagctatgaccatgctctttcagtggttgggttactctc

SEQ ID NO: 284 - DNA, 44 bp, Listeria monocytogenes
gtggaacgacggcgagaccgttccatcaacatcataacttactg